(19) United States
(12) Patent Application Publication
(10) Pub. No.: US 2023/0218581 A1
(43) Pub. Date: Jul. 13, 2023

(54) COMPOSITIONS AND METHODS COMPRISING DENDRIMERS AND THERAPEUTIC AGENTS

(71) Applicant: The Johns Hopkins University, Baltimore, MD (US)

(72) Inventors: Sujatha Kannan, Baltimore, MD (US); Rana Rais, Baltimore, MD (US); Kannan Rangaramanujam, Baltimore, MD (US); Anjali Sharma, Baltimore, MD (US); Barbara Slusher, Baltimore, MD (US); Carolyn Tallon, Baltimore, MD (US); Ajit Thomas, Baltimore, MD (US); Ranjeev Kumar Pandey, Baltimore, MD (US); Jonathan Powell, Baltimore, MD (US)

(21) Appl. No.: 17/997,006

(22) PCT Filed: Apr. 23, 2021
(86) PCT No.: PCT/US2021/028971

§ 371 Date: Oct. 24, 2022

Related U.S. Application Data
(60) Provisional application No. 63/015,118, filed on Apr. 24, 2020.

(51) Int. Cl. A61K 31/4178; A61K 47/59; A61P 25/28; A61P 35/00; A61K 9/00

(52) U.S. Cl. CPC A61K 31/4178; A61K 47/59; A61P 25/28; A61P 35/00; A61K 9/0019; A61K 9/0056

(57) ABSTRACT

Compositions of dendrimers conjugated with one or more therapeutic agents that decrease exosome secretion and methods of use thereof for treating, alleviating, and/or preventing one or more symptoms associated with one or more neurological disease or disorders, cancer, inflammatory diseases, bacterial and viral infections, and other disorders have been developed. Preferably, the therapeutic agents are one or more agents that inhibit or reduce activity and/or quantity of neutral sphingomyelinase 2 (nSMase2) such as small molecule inhibitors of nSMase2. Compositions are particularly suited for reducing Aβ plaque formation, reducing tau propagation, improving cognition, or combinations thereof in a subject with psychiatric and neurological disorders. Compositions are also suited for treating, alleviating, and/or preventing one or more symptoms associated with cancer, bacterial and viral infections, and inflammatory diseases. Methods of treating a human subject having one or more of the diseases and disorders are provided.

### CROSS-REFERENCE TO RELATED APPLICATION

This application claims benefit of U.S. Provisional Application No. 63/015,118, filed Apr. 24, 2020, which, is hereby incorporated herein by reference in its entirety.

### FIELD OF THE INVENTION

The invention is generally in the field of drug delivery, and in particular, methods for delivering drugs bound via dendrimer formulations selectively to sites or regions of neuroinflammation in need thereof.

### BACKGROUND OF THE INVENTION

Alzheimer's disease (AD) is a progressive multifactorial disease, affecting more than 35 million people worldwide, and is the most common cause of late-life dementia. The mean incidence of AD is 1-3% and is associated with an overall prevalence of 10-30% in persons over 65 years of age which, globally, is predicted to nearly double every 20 years. On average, persons will live with AD for 10 years. In the US, approximately 5.4 million people age 65 and older have been diagnosed with AD, and this number is expected to rise as high as 16 million by 2050. Total costs for caring for the more than 5 million persons living with AD is estimated at $200 billion and are projected to rise to $1.1 trillion by 2050. To date, no interventions have demonstrated substantial therapeutic efficacy to prevent, delay or treat AD and several have actually accelerated disease progression.

Research in the field of AD has embraced the complexity of disease pathophysiology, and has enabled a more diverse therapeutic pipeline targeting multiple different aspects of the disease. Therapeutic agents currently available in the clinic, i.e., acetylcholinesterase inhibitors and the NMDA receptor antagonist memantine, only help in the amelioration of symptoms, but do not reduce or inhibit the underlying disease. Recent clinical trials using BACE-1 or γ-secretase inhibitors to inhibit Amyloid beta (Aβ) production, anti-Aβ immunotherapy to clear Aβ from the brain, and compounds designed to address tau-based pathology have not yielded promising results.

In spite of significant efforts, no effective therapeutic agents or treatment methods have been approved to repair, or counteract, the neuronal damage that is the hallmark AD, or the associated cognitive decline or impairment. New disease modifying treatments are sorely needed.

There are many diseases and disorders for which there are few if any effective treatments, and must suffer from debilitating side effects. Examples include many cancers and infectious disease, most of which have as a primary component inflammation.

It is therefore an object of the invention to provide compositions for the treatment or prevention of inflammation generally, as well as for neuronal damage associated with Alzheimer's disease and the associated cognitive decline or impairment.

Therefore, it is an object of the invention to provide compositions that reduce or prevent the pathological processes associated with the development and progression of cancers, infectious diseases and neurological diseases such as Alzheimer's disease, having inflammation as significant contributors to the pathology, and methods of making and using thereof.

It is also an object of the invention to provide compositions for the treatment or prevention of neuronal damage associated with Alzheimer's disease and the associated cognitive decline or impairment, and methods of making and using thereof.

### SUMMARY OF THE INVENTION

Compositions of dendrimers conjugated with therapeutic agents for the treatment of the pathological processes associated with the development and progression of cancers, infectious diseases and neurological diseases such as Alzheimer's disease, having inflammation as significant contributors to the pathology, have been developed.

Compositions including dendrimers coupled or encapsulated with one or more therapeutic agents that decrease exosome secretion, reduce inflammation, such as that present in cancers and in some infectious diseases, reduce Aβ plaque formation, reduce tau propagation, improve cognition, or combinations thereof, and methods of making and using thereof are provided. In some embodiments, the dendrimers are complexed, covalently conjugated, intra-molecularly dispersed, or encapsulated with one or more therapeutic agents.

Preferably, the therapeutic agents are one or more agents that inhibit or reduce activity and/or quantity of neutral sphingomyelinase 2 (nSMase2), for example, one or more inhibitors of nSMase2. Exemplary nSMase2 inhibitors include 2,6-dimethoxy-4-(5-phenyl-4-(thiophen-2-yl)-1H-imidazol-2-yl) phenol (DPTIP), phenyl(R)-(1-(3-(3,4-dimethoxyphenyl)-2,6-dimethylimidazo[1,2-b]pyridazin-8-yl)pyrrolidin-3-yl)-carbamate (PDDC), N,N′-Bis[4-(4,5-dihydro-1H-imidazol-2-yl)phenyl]-3,3′-p-phenylene-bis-acrylamide dihydrochloride (GW4869), and cambinol.

In some embodiments, the dendrimers are generation 4-8 dendrimers, such as generation 4, generation 5, generation 6, generation 7, or generation 8 dendrimers. Exemplary dendrimers include poly(amidoamine) (PAMAM) dendrimers, particularly hydroxyl-terminated PAMAM dendrimers. In preferred embodiments, the dendrimers are covalently conjugated to the one or more therapeutic agents.

Pharmaceutical compositions including the dendrimer composition and one or more pharmaceutically acceptable excipients are also provided. In particular, formulations suitable for parenteral or oral administration including hydrogels, nanoparticles or microparticles, suspensions, powders, tablets, capsules, and solutions, are described.

Methods for treating, alleviating, and/or preventing one or more pathological processes and/or symptoms associated with inflammation, such as that present in cancers and in some infectious diseases, and in neurological disorders such as Alzheimer's disease, for example, reducing Aβ plaque formation, reducing tau propagation, improving cognition, or combinations thereof, in a subject are also provided. The methods include systemically administering to the subject an effective amount of the dendrimer composition to treat, alleviate, and/or prevent one or more pathological processes and/or symptoms associated with the inflammation, cancer, infectious disease or neurological disease such as Alzheimer's disease. Preferably, the compositions decrease exosome secretion in the brain, reduce Aβ plaque formation and/or tau propagation in the brain, improve cognition, or combinations thereof; inhibit or reduce activity and/or quantity of neutral sphingomyelinase 2 in activated microglia; or reduce the concentration of ceramide in the cerebrospinal fluid and/or serum of the subject. The methods can include identifying a subject having one or more biological markers associated with development of AD or dementia, cancer, inflammation or infectious disease. In a preferred embodiment, the dendrimer compositions are administered to a subject that has an increased level of ceramide in the cerebrospinal fluid and/or serum, compared to a healthy control subject. In some embodiments, the methods reduce the quantity of brain and/or serum exosomes, reduce brain and/or serum ceramide levels, reduce serum anti-ceramide IgG, reduce glial activation, reduce total Aβ42 and plaque burden, reduce tau phosphorylation, improve cognition, or combinations thereof in a subject in need thereof. In other embodiments, the methods inhibit activities of neutral sphingomyelinase 2 in activated microglia in the brain of a subject, increase generation of new neurons, or reduce or prevent the rate of neuron loss in a subject, increase the weight of the brain, and/or reduce or prevent the rate of decrease in brain weight of a subject, increase the hippocampal volume, and/or reduce or prevent the rate of decrease of hippocampal volume of a subject. The methods include administering to the subject, preferably those with an increased level of ceramide in the cerebrospinal fluid and/or serum compared to a healthy control subject and/or with Alzheimer's disease, cancer, infectious disease and/or inflammation, an effective amount of the dendrimer compositions orally or parenterally, or intravenously. Methods for treating a cancer in a subject in need thereof include systemically administering to the subject an effective amount of the dendrimer composition to treat cancer to reduce tumor size or inhibit tumor growth. Exemplary cancer include breast cancer, cervical cancer, ovarian cancer, uterine cancer, pancreatic cancer, skin cancer, multiple myeloma, prostate cancer, testicular germ cell tumor, brain cancer, oral cancer, esophagus cancer, lung cancer, liver cancer, renal cell cancer, colorectal cancer, duodenal cancer, gastric cancer, and colon cancer. In some embodiments, the methods further include administering to the subject one or more immune checkpoint modulators selected from the group consisting of PD-1 antagonists, PD-1 ligand antagonists, and CTLA4 antagonists. In some embodiments, the methods further include administering to the subject adoptive T cell therapy, and/or a cancer vaccine. In some embodiments, the methods also include surgery or radiation therapy. The methods include administering to the subject an effective amount of the dendrimer composition to treat cancer orally or parenterally.

Methods for treating or alleviating one or more inflammatory diseases or disorders in a subject in need thereof include administering to the subject an effective amount of the dendrimer composition to treat or alleviate one or more symptoms associated with the one or more inflammatory diseases or disorders. The methods are particularly suited for treating airway inflammation, allergic airway inflammation, atherosclerosis, cerebral ischemia, hepatic ischemia reperfusion injury, myocardial infarction, and sepsis. In some embodiments an amount of the dendrimer composition effective to suppress or inhibit one or more pro-inflammatory cells associated with the one or more inflammatory diseases or disorders is administered. In some embodiments, the dendrimer composition is administered in an amount effective to suppress or inhibit pro-inflammatory cells such as activated macrophages or microglia.

Methods for treating or alleviating one or more bacterial, parasitic or viral infections in a subject in need thereof are also provided. The methods include administering to the subject an effective amount of the dendrimer composition to treat or alleviate one or more symptoms associated with the one or more viral, bacterial or parasitic infections, for example, caused by one or more causative agents such as human immunodeficiency virus (HIV), Zika virus, Hepatitis C, Hepatitis E, Rabies, Langat virus (LGTV), Dengue virus (DENV), cytomegalovirus (HCMV), and Newcastle disease virus (NDV), Epsilon-toxin from Clostridium perfringens, and shiga toxin from Escherichia coli. The methods are suited for treating or alleviating one or more symptoms where the one or more causative agents target or infect activated macrophages/microglia or astrocytes. Typically, the composition is administered in an amount effective to reduce or inhibit replication, load, and/or release, of the infectious agent.

### BRIEF DESCRIPTION OF THE DRAWINGS

FIG. 1 is a bar graph showing ceramide content in cerebrospinal fluid (CSF) from patients with AD versus control individuals.

FIGS. 2A and 2B are line plots showing inhibition of nSMase2 by DPTIP (IC50=30 nM) (FIG. 2A), and its inactive des-hydroxyl analog (IC50>100 μM) (FIG. 2B).

FIG. 3 is a line plot showing the amount of exosomes released by mouse primary glia (EVs/ml) at different concentrations of DPTIP (0 μM to 100 μM).

FIG. 4 is a line graph showing plasma pharmacokinetics of generation 4 (G4) and G6 dendrimers versus DPTIP expressed as the percent of injected dose over a period of 80 hours.

FIGS. 5A and 5B are schematics showing synthesis of Dendrimer-DPTIP (D-DPTIP) conjugates, including the step of modification of DPTIP to attach an orthogonal linker with azide terminus through a cleavable ester bond (FIG. 5A), and modification of a dendrimer surface to attach a linker bearing complimentary alkyne groups, thus enabling highly efficient copper (I) catalyzed alkyne-azide click (CuAAC) chemistry to produce D-DPTIP conjugates (FIG. 5B).

FIG. 6 is a line plot showing percentage of DPTIP from D-DPTIP conjugates over a period of 600 hours in vitro in the presence of esterase (pH 5.5) at physiological temperature.

FIG. 7 is a bar graph showing SMnase2 activity (RFU/mg/h) in glial cells in the brains of vehicle-treated group and D-DPTIP treated group following peroral administration.

FIGS. 8A and 8B are bar graphs showing concentrations of DPTIP (pmol/ml or pmol/g) from D-DPTIP in the plasma (FIG. 8A) and in the brain (FIG. 8B) at 24 hours, 72 hours and 120 hours post oral administration of D-DPTIP at 10 mg/kg, 30 mg/kg and 100 mg/kg free drug equivalent.

FIGS. 9A and 9B are bar graphs showing nSMase 2 activity (RFU/mg/h) in brain microglial cells (FIG. 9A) and non-microglial cells (FIG. 9B) from hTau-injected PS19 (AD) mice following oral administration of vehicle, 10 mg/kg D-DPTIP, or 100 mg/kg D-DPTIP, and in mice with no hTau injected (uninjected).

FIG. 10 is a line graph showing tumor volume (mm³) over 28 days post M38 injection in six- to eight-week-old male C57BL/6 mice treated by i.p. injection with Isotype Control, Anti-PDL1, D-DPTIP Control, or D-DPTIP in combination with Anti-PDL1 (Anti-PDL1+D-DPTIP).

FIG. 11 is a bar graph showing concentrations of DPTIP (pmol/ml or pmol/g) in from D-DPTIP in the plasma (FIG. 11A) and in the tumor (FIG. 11B) at 6 hours, 24 hours, and 48 hours post administration of D-DPTIP at 10 mg/kg free drug (DPTIP) equivalent.

FIG. 12 is a bar graph showing mean fluorescence intensity (MFI) in neurons of the contralateral/ipsilateral dentate gyms (DG) in vehicle treated or D-DPTIP treated mice.

### DETAILED DESCRIPTION OF THE INVENTION

### I. Definitions

The terms “active agent” or “biologically agent” are therapeutic, prophylactic or diagnostic agents used interchangeably to refer to a chemical or biological compound that induces a desired pharmacological and/or physiological effect, which may be prophylactic, therapeutic or diagnostic. These may be a nucleic acid, a nucleic acid analog, a small molecule having a molecular weight less than 2 kD, more typically less than 1 kD, a peptidomimetic, a protein or peptide, carbohydrate or sugar, lipid, or a combination thereof. The terms also encompass pharmaceutically acceptable, pharmacologically active derivatives of agents, including, but not limited to, salts, esters, amides, prodrugs, active metabolites, and analogs.

The term “analog” refers to a chemical compound with a structure similar to that of another (reference compound) but differing from it in respect to a particular component, functional group, atom, etc.

The term “derivative” refers to compounds which are formed from a parent compound by one or more chemical reaction(s).

The term “pharmaceutically acceptable salts” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine;

The term “therapeutic agent” refers to an agent that can be administered to treat one or more symptoms of a disease or disorder.

The term “diagnostic agent” generally refers to an agent that can be administered to reveal, pinpoint, and define the localization of a pathological process. The diagnostic agents can label target cells that allow subsequent detection or imaging of these labeled target cells. In some embodiments, diagnostic agents can, via dendrimer or suitable delivery vehicles, target/bind activated microglia in the central nervous system (CNS).

The term “prophylactic agent” generally refers to an agent that can be administered to prevent disease or to prevent certain conditions, such as a vaccine.

The phrase “pharmaceutically acceptable” or “biocompatible” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “therapeutically effective amount” refers to an amount of the therapeutic agent that, when incorporated into and/or onto dendrimers, produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish the symptoms of one or more diseases or disorders, such as reducing, preventing, or reversing the learning and/or memory deficits in an individual suffering from Alzheimer's disease etc. In one or more neurological or neurodegenerative diseases, an effective amount of the drug may have the effect of stimulation or induction of neural mitosis leading to the generation of new neurons, i.e., exhibiting a neurogenic effect; prevention or retardation of neural loss, including a decrease in the rate of neural loss, i.e., exhibiting a neuroprotective effect. An effective amount can be administered in one or more administrations.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%. For example, dendrimer compositions including one or more inhibitors may inhibit or reduce the activity and/or quantity of nSMase2 associated activated microglia by about 10%, 20%, 30%, 40%, 50%, 75%, 85%, 90%, 95%, or 99% from the activity and/or quantity of the same cells in equivalent tissues of subjects that did not receive, or were not treated with the dendrimer compositions. In some embodiments, the inhibition and reduction are compared at mRNAs, proteins, cells, tissues and organs levels. For example, an inhibition and reduction in the rate of neural loss, in the rate of decrease of brain weight, or in the rate of decrease of hippocampal volume, as compared to an untreated control subject.

The term “treating” or “preventing” a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating or palliating the disease state, and remission or improved prognosis. For example, an individual is successfully “treated” if one or more symptoms associated with Alzheimer's disease are mitigated or eliminated, including, but are not limited to, reducing the rate of neuronal loss, decreasing symptoms resulting from the disease, increasing the quality of life of those suffering from the disease, decreasing the dose of other medications required to treat the disease, delaying the progression of the disease, and/or prolonging survival of individuals.

The term “biodegradable” generally refers to a material that will degrade or erode under physiologic conditions to smaller units or chemical species that are capable of being metabolized, eliminated, or excreted by the subject. The degradation time is a function of composition and morphology.

The term “dendrimer” includes, but is not limited to, a molecular architecture with an interior core, interior layers (or “generations”) of repeating units regularly attached to this initiator core, and an exterior surface of terminal groups attached to the outermost generation.

The term “functionalize” means to modify a compound or molecule in a manner that results in the attachment of a functional group or moiety. For example, a molecule may be functionalized by the introduction of a molecule that makes the molecule a strong nucleophile or strong electrophile.

The term “targeting moiety” refers to a moiety that localizes to or away from a specific locale. The moiety may be, for example, a protein, nucleic acid, nucleic acid analog, carbohydrate, or small molecule. The entity may be, for example, a therapeutic compound such as a small molecule, or a diagnostic entity such as a detectable label. The locale may be a tissue, a particular cell type, or a subcellular compartment. In one embodiment, the targeting moiety directs the localization of an agent. In preferred embodiment, the dendrimer composition can selectively target activated microglia in the absence of an additional targeting moiety.

The term “prolonged residence time” refers to an increase in the time required for an agent to be cleared from a patient's body, or organ or tissue of that patient. In certain embodiments, “prolonged residence time” refers to an agent that is cleared with a half-life that is 10%, 20%, 50% or 75% longer than a standard of comparison such as a comparable agent without conjugation to a delivery vehicle such as a dendrimer. In certain embodiments, “prolonged residence time” refers to an agent that is cleared with a half-life of 2, 5, 10, 20, 50, 100, 200, 500, 1000, 2000, 5000, or 10000 times longer than a standard of comparison such as a comparable agent without a dendrimer that specifically target specific cell types.

The terms “incorporated” and “encapsulated” refer to incorporating, formulating, or otherwise including an agent into and/or onto a composition that allows for release, such as sustained release, of such agent in the desired application. The agent or other material can be incorporated into a dendrimer, by binding to one or more surface functional groups of such dendrimer (by covalent, ionic, or other binding interaction), by physical admixture, by enveloping the agent within the dendritic structure, and/or by encapsulating the agent inside the dendritic structure.

### II. Compositions

Dendrimer complexes suitable for delivering one or more agent, particularly one or more agents to prevent, treat or diagnose one or more neurological and neurodegenerative diseases, especially dementia, cancer, infectious disease, and other disorders associated with inflammation have been developed.

Compositions of dendrimer complexes include one or more prophylactic, therapeutic, and/or diagnostic agents encapsulated, associated, and/or conjugated with the dendrimers. Generally, one or more agent is encapsulated, associated, and/or conjugated in the dendrimer complex at a concentration of about 0.01% to about 30%, preferably about 1% to about 20%, more preferably about 5% to about 20% by weight. Preferably, an agent is covalently conjugated to the dendrimer via one or more linkages such as disulfide, ester, ether, thioester, carbamate, carbonate, hydrazine, and amide, optionally via one or more spacers. In some embodiments, the spacer is an agent, such as N-acetyl cysteine. Exemplary agents include anti-inflammatory drugs, chemotherapeutics, anti-seizure agents, vasodilators, and anti-infective agents.

The presence of the additional agents can affect the zeta-potential or the surface charge of the particle. In one embodiment, the zeta potential of the dendrimers is between −100 mV and 100 mV, between −50 mV and 50 mV, between −25 mV and 25 mV, between −20 mV and 20 mV, between −10 mV and 10 mV, between −10 mV and 5 mV, between −5 mV and 5 mV, or between −2 mV and 2 mV. In a preferred embodiment, the surface charge is neutral or near-neutral. The range above is inclusive of all values from −100 mV to 100 mV.

A. Dendrimers

Dendrimers are three-dimensional, hyperbranched, monodispersed, globular and polyvalent macromolecules comprising a high density of surface end groups (Tomalia, D. A., et al., Biochemical Society Transactions, 35, 61 (2007); and Sharma, A., et al., ACS Macro Letters, 3, 1079 (2014)). Due to their unique structural and physical features, dendrimers are useful as nanocarriers for various biomedical applications including targeted drug/gene delivery, imaging and diagnosis (Sharma, A., et al., RSC Advances, 4, 19242 (2014); Caminade, A.-M., et al., Journal of Materials Chemistry B, 2, 4055 (2014); Esfand, R., et al., Drug Discovery Today, 6, 427 (2001); and Kannan, R. M., et al., Journal of Internal Medicine, 276, 579 (2014)).

Dendrimer surface groups have a significant impact on their biodistribution (Nance, E., et al., Biomaterials, 101, 96 (2016)). Hydroxyl terminated generation 4 PAMAM dendrimers (˜4 nm size) without any targeting ligand cross the impaired BBB upon systemic administration in a rabbit model of cerebral palsy (CP) significantly more (>20 fold) as compared to healthy controls, and selectively target activated microglia and astrocytes (Lesniak, W. G., et al., Mol Pharm, 10 (2013)).

The term “dendrimer” includes, but is not limited to, a molecular architecture with an interior core and layers (or “generations”) of repeating units which are attached to and extend from this interior core, each layer having one or more branching points, and an exterior surface of terminal groups attached to the outermost generation. In some embodiments, dendrimers have regular dendrimeric or “starburst” molecular structures.

Generally, dendrimers have a diameter between about 1 nm and about 50 nm, more preferably between about 1 nm and about 20 nm, between about 1 nm and about 10 nm, or between about 1 nm and about 5 nm. In some embodiments, the diameter is between about 1 nm and about 2 nm. Conjugates are generally in the same size range, although large proteins such as antibodies may increase the size by 5-15 nm. In general, agent is encapsulated in a ratio of agent to dendrimer of between 1:1 and 4:1 for the larger generation dendrimers. In preferred embodiments, the dendrimers have a diameter effective to penetrate brain tissue and to retain in target cells for a prolonged period of time.

In some embodiments, dendrimers have a molecular weight between about 500 Daltons and about 100,000 Daltons, preferably between about 500 Daltons and about 50,000 Daltons, most preferably between about 1,000 Daltons and about 20,000 Dalton.

Suitable dendrimers scaffolds that can be used include poly(amidoamine) dendrimers, also known as PAMAM, or STARBURST™ dendrimers; polypropylamine (POPAM), polyethylenimine, polylysine, polyester, iptycene, aliphatic poly(ether), and/or aromatic polyether dendrimers. The dendrimers can have carboxylic, amine and/or hydroxyl terminations. The dendrimer may have all or a percentage of these terminations. In a preferred embodiment, the dendrimer is primarily hydroxyl terminated. Each dendrimer of the dendrimer complex may be same or of similar or different chemical nature than the other dendrimers (e.g., the first dendrimer may include a PAMAM dendrimer, while the second dendrimer may be a POPAM dendrimer).

The term “PAMAM dendrimer” means poly(amidoamine) dendrimer, which may contain different cores, with amidoamine building blocks, and can have carboxylic, amine and hydroxyl terminations of any generation including, but not limited to, generation 1 PAMAM dendrimers, generation 2 PAMAM dendrimers, generation 3 PAMAM dendrimers, generation 4 PAMAM dendrimers, generation 5 PAMAM dendrimers, generation 6 PAMAM dendrimers, generation 7 PAMAM dendrimers, generation 8 PAMAM dendrimers, generation 9 PAMAM dendrimers, or generation 10 PAMAM dendrimers. In the preferred embodiment, the dendrimers are soluble in the formulation and are generation (“G”) 4, 5 or 6 dendrimers. The dendrimers may have hydroxyl groups attached to their functional surface groups.

Methods for making dendrimers are known to those of skill in the art and generally involve a two-step iterative reaction sequence that produces concentric shells (generations) of dendritic β-alanine units around a central initiator core (e.g., ethylenediamine-cores). Each subsequent growth step represents a new “generation” of polymer with a larger molecular diameter, twice the number of reactive surface sites, and approximately double the molecular weight of the preceding generation. Dendrimer scaffolds suitable for use are commercially available in a variety of generations. Preferable, the dendrimer compositions are based on generation 0, 1, 2, 3, 4, 5, 6, 7, 8, 9, or 10 dendrimeric scaffolds. Such scaffolds have, respectively, 4, 8, 16, 32, 64, 128, 256, 512, 1024, 2048, and 4096 reactive sites. Thus, the dendrimeric compounds based on these scaffolds can have up to the corresponding number of combined targeting moieties, if any, and agents.

In some embodiments, the dendrimers include a plurality of hydroxyl groups. Some exemplary high-density hydroxyl groups-containing dendrimers include commercially available polyester dendritic polymer such as hyperbranched 2,2-Bis(hydroxyl-methyl)propionic acid polyester polymer (for example, hyperbranched bis-MPA polyester-64-hydroxyl, generation 4), dendritic polyglycerols.

In some embodiments, the high-density hydroxyl groups-containing dendrimers are oligo ethylene glycol (OEG)-like dendrimers. For example, a generation 2 OEG dendrimer (D2-OH-60) can be synthesized using highly efficient, robust and atom economical chemical reactions such as Cu (I) catalyzed alkyne-azide click and photo catalyzed thiol-ene click chemistry. Highly dense polyol dendrimer at very low generation in minimum reaction steps can be achieved by using an orthogonal hypermonomer and hypercore strategy, for example as described in International Patent Publication No. WO2019094952. In some embodiments, the dendrimer backbone has non-cleavable polyether bonds throughout the structure to avoid the disintegration of dendrimer in vivo and to allow the elimination of such dendrimers as a single entity from the body (non-biodegradable).

In some embodiments, the dendrimer specifically targets a particular tissue region and/or cell type, preferably activated macrophages in the CNS. In preferred embodiments, the dendrimer specifically targets a particular tissue region and/or cell type without a targeting moiety.

In preferred embodiments, the dendrimers have a plurality of hydroxyl (—OH) groups on the periphery of the dendrimers. The preferred surface density of hydroxyl (—OH) groups is at least 1 OH group/nm² (number of hydroxyl surface groups/surface area in nm²). For example, in some embodiments, the surface density of hydroxyl groups is more than 2, 3, 4, 5, 6, 7, 8, 9, 10; preferably at least 10, 15, 20, 25, 30, 35, 40, 45, 50, or more than 50. In further embodiments, the surface density of hydroxyl (—OH) groups is between about 1 and about 50, preferably 5-20 OH group/nm² (number of hydroxyl surface groups/surface area in nm²) while having a molecular weight of between about 500 Da and about 10 kDa.

In some embodiments, the dendrimers may have a fraction of the hydroxyl groups exposed on the outer surface, with the others in the interior core of the dendrimers. In preferred embodiments, the dendrimers have a volumetric density of hydroxyl (—OH) groups of at least 1 OH group/nm³ (number of hydroxyl groups/volume in nm³). For example, in some embodiments, the volumetric density of hydroxyl groups is 2, 3, 4, 5, 6, 7, 8, 9, 10, or more than 10, 15, 20, 25, 30, 35, 40, 45, and 50. In some embodiments, the volumetric density of hydroxyl groups is between about 4 and about 50 groups/nm³, preferably between about 5 and about 30 groups/nm³, more preferably between about 10 and about 20 groups/nm³.

B. Coupling Agents and Spacers

Dendrimer complexes can be formed of therapeutically agents or compounds conjugated or attached to a dendrimer, a dendritic polymer or a hyperbranched polymer. Optionally, the agents are conjugated to the dendrimers via one or more spacers/linkers via different linkages such as disulfide, ester, carbonate, carbamate, thioester, hydrazine, hydrazides, and amide linkages. The one or more spacers/linkers between a dendrimer and an agent can be designed to provide a releasable or non-releasable form of the dendrimer-active complexes in vivo. In some embodiments, the attachment occurs via an appropriate spacer that provides an ester bond between the agent and the dendrimer. In some embodiments, the attachment occurs via an appropriate spacer that provides an amide bond between the agent and the dendrimer. In preferred embodiments, one or more spacers/linkers between a dendrimer and an agent are added to achieve desired and effective release kinetics in vivo.

The term “spacers” includes compositions used for linking a therapeutically agent to the dendrimer. The spacer can be either a single chemical entity or two or more chemical entities linked together to bridge the polymer and the therapeutic agent or imaging agent. The spacers can include any small chemical entity, peptide or polymers having sulfhydryl, thiopyridine, succinimidyl, maleimide, vinylsulfone, and carbonate terminations.

The spacer can be chosen from among a class of compounds terminating in sulfhydryl, thiopyridine, succinimidyl, maleimide, vinylsulfone and carbonate group. The spacer can include thiopyridine terminated compounds such as dithiodipyridine, N-Succinimidyl 3-(2-pyridyldithio)-propionate (SPDP), Succinimidyl 6-(3-[2-pyridyldithio]-propionamido)hexanoate LC-SPDP or Sulfo-LC-SPDP. The spacer can also include peptides wherein the peptides are linear or cyclic essentially having sulfhydryl groups such as glutathione, homocysteine, cysteine and its derivatives, arg-gly-asp-cys (RGDC), cyclo(Arg-Gly-Asp-d-Phe-Cys) (c(RGDfC)), cyclo(Arg-Gly-Asp-D-Tyr-Cys), cyclo(Arg-Ala-Asp-d-Tyr-Cys). The spacer can be a mercapto acid derivative such as 3 mercapto propionic acid, mercapto acetic acid, 4 mercapto butyric acid, thiolan-2-one, 6 mercaptohexanoic acid, 5 mercapto valeric acid and other mercapto derivatives such as 2 mercaptoethanol and 2 mercaptoethylamine The spacer can be thiosalicylic acid and its derivatives, (4-succinimidyloxycarbonyl-methyl-alpha-2-pyridylthio)toluene, (3[2-pyridithio]propionyl hydrazide, The spacer can have maleimide terminations wherein the spacer includes polymer or small chemical entity such as bis-maleimido diethylene glycol and bis-maleimido triethylene glycol, Bis-Maleimidoethane, bismaleimidohexane. The spacer can include vinylsulfone such as 1,6-Hexane-bis-vinylsulfone. The spacer can include thioglycosides such as thioglucose. The spacer can be reduced proteins such as bovine serum albumin and human serum albumin, any thiol terminated compound capable of forming disulfide bonds. The spacer can include polyethylene glycol having maleimide, succinimidyl and thiol terminations.

The agent and/or targeting moiety can be either covalently attached or intra-molecularly dispersed or encapsulated. The dendrimer is preferably a PAMAM dendrimer up to generation 10, having carboxylic, hydroxyl, or amine terminations. In preferred embodiments, the dendrimer is linked to agents via a spacer ending in disulfide, ester or amide bonds.

C. Therapeutic, Prophylactic and Diagnostic, Agents

Agents to be included in the particles to be delivered can be proteins or peptides, sugars or carbohydrate, nucleic acids or oligonucleotides, lipids, small molecules (e.g., molecular weight less than 2000 Dalton, preferably less than 1500 Dalton, more preferably 300-700 Dalton), or combinations thereof. The nucleic acid can be an oligonucleotide encoding a protein, for example, a DNA expression cassette or an mRNA. Representative oligonucleotides include siRNAs, microRNAs, DNA, and RNA. In some embodiments, the agent is a therapeutic antibody.

Dendrimers have the advantage that multiple therapeutic, prophylactic, and/or diagnostic agents can be delivered with the same dendrimers. One or more types of agents can be encapsulated, complexed or conjugated to the dendrimer. In one embodiment, the dendrimers are complexed with or conjugated to two or more different classes of agents, providing simultaneous delivery with different or independent release kinetics at the target site. In another embodiment, the dendrimers are covalently linked to at least one detectable moiety and at least one class of agents. In a further embodiment, dendrimer complexes each carrying different classes of agents are administered simultaneously for a combination treatment.

Therapeutic or prophylactic agents can include those agents that manipulate enzymatic or receptor-mediated mechanisms in activated microglia for the treatment of one or more neurological diseases. Exemplary enzymatic or receptor-mediated mechanisms include, but not limited to, those of neutral sphingomyelinase 2 (nSMase2), triggering receptor expressed on myeloid cells 2 (TREM2), leucine-rich repeat kinase 2 (LRRK2), and receptor-interacting serine/threonine-protein kinase 1 (RIPK1). In some embodiments, the agents are those that can restore altered activities in the enzymatic or receptor-mediated mechanisms involving one or more of nSMase2, TREM2, LRRK2, and RIPK1.

1. Neutral Sphingomyelinase Inhibitors

Both Aβ aggregation and tau protein propagation, two major hallmarks of Alzheimer's disease, involve exosome secretion. Exosomes are small extracellular vesicles (EVs) carrying protein, lipid and RNA that are shed from cells in response to various stimuli. Under several neurological disease conditions, EVs can carry pathological cargo and play an active role in disease progression. The brain enzyme neutral sphingomyelinase 2 (nSMase2), is a critical regulator of EV biogenesis through its production of ceramide, which is a major EV component and thus represents a unique AD therapeutic target. Pharmacological inhibition and genetic deletion of nSMase2 has been shown to reduce brain ceramide and decrease EV secretion, reduce Aβ plaque formation and tau propagation, and improve cognition in mouse models of AD. Thus, nSMase inhibition represents a therapeutic approach for treatment of AD and other neurological diseases.

Ceramide is essential for the biogenesis of exosomes and that pharmacological inhibition of nSMase2 reduced exosome secretion. nSMase2 catalyzes the hydrolysis of sphingomyelin (SM) to phosphorylcholine and ceramide. Production of ceramide through nSMase2 activation has been associated with diverse functions ranging from apoptosis to modulation of synaptic plasticity to manufacturing of ceramide-rich exosomes. While transient nSMase2 activation is part of normal brain functioning, chronic activation of the enzyme results in negative effects including neurodegeneration. Specifically, increased nSMase2 activity has been associated with altered sphingolipid metabolism, neuronal apoptosis, chronic inflammation, and oxidative stress.

Chronic activation of nSMase2 has been reported to be associated with the pathogenesis of HIV-associated dementia (HAD), multiple sclerosis (MS), and amyotrophic lateral sclerosis (ALS). There is evidence that associates chronic increase of nSMase2 activity with AD in human and animal. There are three mammalian nSMases identified to date: nSMase1, nSMase2, and nSMase3. They all catalyze the hydrolysis of sphingomyelin (SM) to phosphorylcholine and ceramide in cell-free biochemical assays, although the physiological roles of nSMase1 and 3 have been harder to elucidate than for nSMase2. Even though nSMase1 can hydrolyze SM in vitro, cell lines over-expressing nSMase1 did not exhibit changes in SM metabolism. nSMase3 has a low sequence identity to the other two nSMases and it is possible that it serves a different function. In contrast, nSMase2 has been shown to have an impact on SM metabolism in cells, and its chronic activation has been specifically implicated in the pathogenesis of neurodegenerative disorders. nSMase2 is predominantly expressed in the CNS (Fensome A C et al. J Biol Chem. 2000; 275(2):1128-36; Hofmann K et al., Proc Natl Acad Sci USA. 2000; 97(11):5895-900; Clarke C J et al., Biochim Biophys Acta. 2006; 1758(12):1893-901). nSMase2 is primarily located on the Golgi apparatus, but can translocate to perinuclear regions in response to the antioxidant glutathione and to the inner leaflet of the plasma membrane in response to oxidative stress.

Pharmacological inhibition of nSMase2 activity was highly effective in slowing tau propagation in vivo (Asai H et al., Nat Neurosci. 2015; 18(11):1584-93). In one model, following tau propagation from the entorhinal cortex to the dentate gyrus (DG), the prototype nSMase2 inhibitor GW4869 suppressed the number of AT8+ granular neurons (i.e., neurons recognized by monoclonal antibody specific to tau phosphorylation) in the dentate gyrus by 75%. The number of AT8+ cells in the dentate gyms was also reduced, demonstrating the involvement of exosome synthesis in tau transmission. In the second model, nSMase2 inhibition treatment of P301S/PS19 tau mice significantly reduced the number of AT8+ cells in the granular cell layer (GCL) of the DG by 52% but not in the medial entorhinal cortex (MEC). Consistent with these data, dot blot analysis using T22 antibody revealed a significant reduction in tau oligomer accumulation in hippocampal but not EC regions in the inhibitor-treated group. In other in vitro experiment, the specific role of activated glial cells was studied. It was shown that silencing nSMase2 expression or inhibiting nSMase2 activity in LPS/ATP activated microglia significantly reduced secretion of hTau in exosomes. Moreover, treatment of primary cultured neurons with tau-containing exosomal fraction from microglia treated with nSMase2 siRNA or GW4869 showed 70 and 68% reduced transduction of hTau into neurons compared to control groups, respectively (Asai H et al., Nat Neurosci. 2015; 18(11):1584-93).

Additional studies showed that exosomes could stimulate Aβ aggregation in the 5XFAD mouse model of AD (Dinkins M B et al., Neurobiol Aging. 2014; 35(8):1792-800). Further, inhibition of exosome secretion with the prototype nSMase2 inhibitor GW4869 resulted in reduced levels of brain and serum exosomes, brain ceramide, and Aβ plaque load. In a more recent study, also using 5XFAD mice, nSMase2 deficiency alleviated AD pathology and improved cognition; compared to regular 5XFAD mice, nSMase2 deficient 5XFAD mice exhibited reduced brain exosomes, ceramide levels, serum anticeramide IgG, glial activation, total Aβ and plaque burden, tau phosphorylation and improved cognition in a fear conditioned learning task.

In summary, results using three murine AD models show that nSMase2 is involved in both Aβ plaque aggregation and tau propagation. Moreover, pharmacological inhibition or genetic deletion of nSMase2 results in improvements in pathological measures and cognitive outcomes.

Accordingly, in some embodiments, the dendrimer compositions include one or more therapeutic agents that decrease exosome secretion, reduce Aβ plaque formation, reduce tau propagation, improve cognition, or combinations thereof. In some embodiments, the dendrimer compositions include one or more therapeutic agents that inhibit or reduce activity and/or quantity of nSMase2. In some embodiments, the dendrimer compositions include one or more neutral sphingomyelinase inhibitors. In some embodiments, the dendrimer compositions include one or more small molecule neutral sphingomyelinase inhibitors having molecular weight less than 2000 Dalton, preferably less than 1500 Dalton. In some embodiments, the one or more neutral sphingomyelinase inhibitors are functionalized, for example, with ether, ester, or amide linkage, optionally with one or more spacers/linkers, for ease of conjugation with the dendrimers and/or for desired release kinetics. In preferred embodiments, the dendrimers are generation 4, generation 5, or generation 6 hydroxyl-terminated PAMAM dendrimer.

In one embodiment, the neutral sphingomyelinase inhibitor is 2,6-dimethoxy-4-(5-phenyl-4-(thiophen-2-yl)-1H-imidazol-2-yl) phenol (DPTIP), or analogs thereof. Analogs of DPTIP have been described previously, for example, in WO2019169247A1. The chemical structure of DPTIP is shown in structure I:

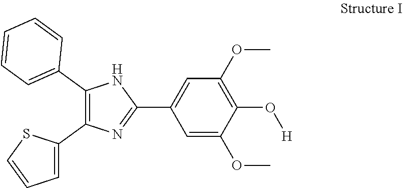 [SMILES: COc1cc(-c2nc(-c3cccs3)c(-c3ccccc3)[nH]2)cc(OC)c1O]

In some embodiments, the neutral sphingomyelinase inhibitor is a 4-(1H-imidazol-2-yl)-2,6-dialkoxyphenol derivative including compounds based on the 4-(1H-imidazol-2-yl)-2,6-dialkoxyphenol scaffold such as those described in Stepanek O et al., Eur J Med Chem. 2019 May 15; 170:276-289, which is specifically incorporated by reference herein in its entirety. In one embodiment, the neutral sphingomyelinase inhibitor is 4-(4,5-diisopropyl-1H-imidazol-2-yl)-2,6-dimethoxyphenol.

Phenyl(R)-(1-(3-(3,4-dimethoxyphenyl)-2,6-dimethylimidazo[1,2-b]pyridazin-8-yl)pyrrolidin-3-yl)-carbamate (PDDC) is a potent (pIC50=6.57) and selective non-competitive inhibitor of nSMase2, as described in Rojas C et al., Br J Pharmacol. 2019 October; 176(19):3857-3870. Accordingly, in one embodiment, the neutral sphingomyelinase inhibitor is phenyl(R)-(1-(3-(3,4-dimethoxyphenyl)-2,6-dimethylimidazo[1,2-b]pyridazin-8-yl)pyrrolidin-3-yl)-carbamate (PDDC), or analogs thereof. The chemical structure of PDDC is shown in structure II.

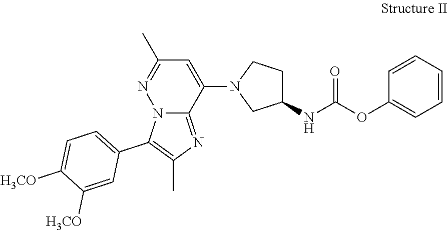 [SMILES: COc1ccc(-c2c(C)nc3c(N4CC[C@@H](NC(=O)Oc5ccccc5)C4)cc(C)nn23)cc1OC]

In another embodiment, the neutral sphingomyelinase inhibitor is cambinol. The chemical structure of cambinol is shown in structure III.

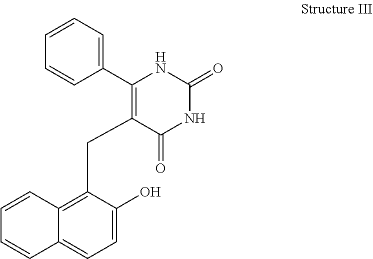 [SMILES: O=c1[nH]c(-c2ccccc2)c(Cc2c(O)ccc3ccccc23)c(=O)[nH]1]

In a further embodiment, the neutral sphingomyelinase inhibitor is N,N′-Bis[4-(4,5-dihydro-1H-imidazol-2-yl)phenyl]-3,3′-p-phenylene-bis-acrylamide dihydrochloride (GW4869). The chemical structure of GW4869 is shown in structure IV.

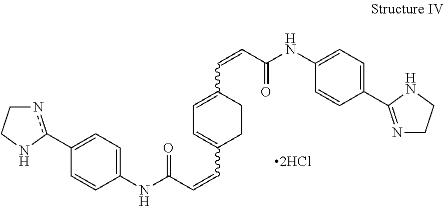

In some embodiments, the neutral sphingomyelinase inhibitor is one or more of structures V-VIII shown below:

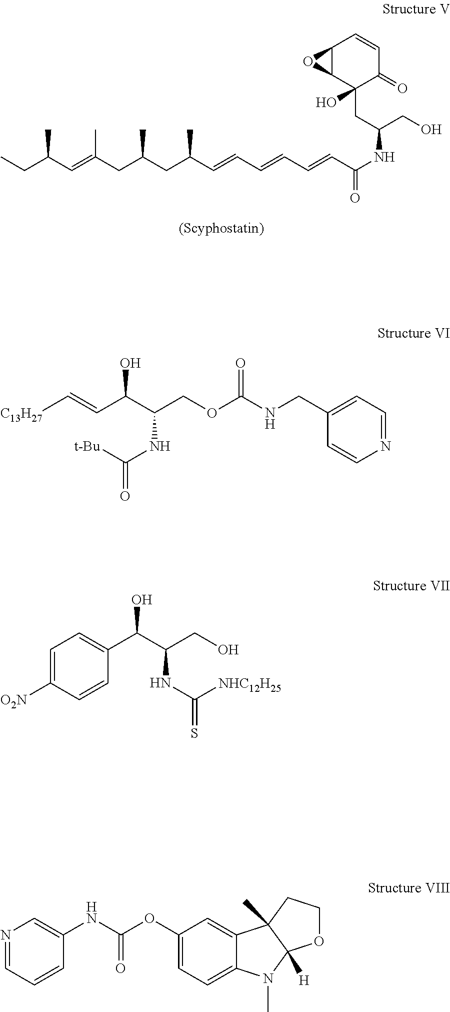

D. Additional Therapeutic and Prophylactic Agents to be Delivered

The dendrimers can be used to deliver one or more additional therapeutic or prophylactic agents, particularly one or more agents to prevent or treat one or more symptoms of the neurological or neurodegenerative diseases, cancer, infectious disease and/or inflammation. Suitable therapeutic, diagnostic, and/or prophylactic agents can be a biomolecule, such as peptides, proteins, carbohydrates, nucleotides or oligonucleotides, or a small molecule agent (e.g., molecular weight less than 2000 amu, preferably less than 1500 amu), including organic, inorganic, and organometallic agents. The agent can be encapsulated within the dendrimers, dispersed within the dendrimers, and/or associated with the surface of the dendrimer, either covalently or non-covalently.

1. Therapeutic and Prophylactic Agents

The dendrimer complexes include one or more therapeutic, prophylactic, or prognostic agents that are complexed or conjugated to the dendrimers. Representative therapeutic agents include, but are not limited to, neuroprotective agents, anti-inflammatory agents, antioxidants, anti-infectious agents, and combinations thereof.

In one embodiment, the additional agent is a steroid. Suitable steroids include biologically active forms of vitamin D3 and D2, such as those described in U.S. Pat. Nos. 4,897,388 and 5,939,407. The steroids may be co-administered to further aid in neurogenic stimulation or induction and/or prevention of neural loss, particularly for treatments of Alzheimer's disease. Estrogen and estrogen related molecules such as allopregnanolone can be co-administered with the neuro-enhancing agents to enhance neuroprotection as described in Brinton (2001) Learning and Memory 8 (3): 121-133.

Other neuroactive steroids, such as various forms of dehydroepiandrosterone (DHEA) as described in U.S. Pat. No. 6,552,010, can also be co-administered to further aid in neurogenic stimulation or induction and/or prevention of neural loss. Other agents that cause neural growth and outgrowth of neural networks, such as Nerve Growth Factor (NGF) and Brain-derived Neurotrophic Factor (BDNF), can be administered either simultaneously with or before or after the administration of THP. Additionally, inhibitors of neural apoptosis, such as inhibitors of calpains and caspases and other cell death mechanisms, such as necrosis, can be co-administered with the neuro-enhancing agents to further prevent neural loss associated with certain neurological diseases and neurological defects.

Representative small molecules include steroids such as methyl prednisone, dexamethasone, non-steroidal anti-inflammatory agents, including COX-2 inhibitors, corticosteroid anti-inflammatory agents, gold compound anti-inflammatory agents, immunosuppressive, anti-inflammatory and anti-angiogenic agents, anti-excitotoxic agents such as valproic acid, D-aminophosphonovalerate, D-aminophosphonoheptanoate, inhibitors of glutamate formation/release, baclofen, NMDA receptor antagonists, salicylate anti-inflammatory agents, ranibizumab, anti-VEGF agents, including aflibercept, and rapamycin. Other anti-inflammatory drugs include nonsteroidal drug such as indomethacin, aspirin, acetaminophen, diclofenac sodium and ibuprofen. The corticosteroids can be fluocinolone acetonide and methylprednisolone.

Representative oligonucleotides include siRNAs, microRNAs, DNA, and RNA.

2. Diagnostic Agents

In some cases, the agent is a diagnostic, alone or in combination with other therapeutic or prophylactic agents. Examples of diagnostic agents include paramagnetic molecules, fluorescent compounds, magnetic molecules, and radionuclides, x-ray imaging agents, and contrast media. Examples of other suitable contrast agents include gases or gas emitting compounds, which are radioopaque. Dendrimer complexes can further include agents useful for determining the location of administered compositions. Agents useful for this purpose include fluorescent tags, radionuclides and contrast agents.

Exemplary diagnostic agents include dyes, fluorescent dyes, near infra-red dyes, SPECT imaging agents, PET imaging agents and radioisotopes. Representative dyes include carbocyanine, indocarbocyanine, oxacarbocyanine, thüicarbocyanine and merocyanine, polymethine, coumarine, rhodamine, xanthene, fluorescein, boron-dipyrromethane (BODIPY), Cy5, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800RS, IRDye 700DX, ADS780WS, ADS830WS, and ADS832WS.

Exemplary SPECT or PET imaging agents include chelators such as di-ethylene tri-amine penta-acetic acid (DTPA), 1,4,7,10-tetra-azacyclododecane-1,4,7,10-tetraacetic acid (DOTA), di-amine dithiols, activated mercaptoacetyl-glycyl-glycyl-glycine (MAG3), and hydrazidonicotinamide (HYNIC).

Exemplary isotopes include Tc-94m, Tc-99m, In-111, Ga-67, Ga-68, Gd3+, Y-86, Y-90, Lu-177, Re-186, Re-188, Cu-64, Cu-67, Co-55, Co-57, F-18, Sc-47, Ac-225, Bi-213, Bi-212, Pb-212, Sm-153, Ho-166, and Dy-166.

In preferred embodiments, the dendrimer complex includes one or more radioisotopes suitable for positron emission tomography (PET) imaging. Exemplary positron-emitting radioisotopes include carbon-11 (¹¹C), copper-64 (⁶⁴Cu), nitrogen-13 (¹³N), oxygen-15 (¹⁵O), gallium-68 (⁶⁸Ga), and fluorine-18 (¹⁸F), e.g., 2-deoxy-2-¹⁸F-fluoro-β-D-glucose (¹⁸F-FDG).

In further embodiments, a singular dendrimer complex composition can simultaneously treat and/or diagnose a disease or a condition at one or more locations in the body.

### III. Pharmaceutical Formulations

Pharmaceutical compositions including dendrimers and one or more inhibitors of sphingomyelin e.g., nSMase2, may be formulated in a conventional manner using one or more physiologically acceptable carriers. Proper formulation is dependent upon the route of administration chosen. In preferred embodiments, the compositions are formulated for parenteral delivery. In some embodiments, the compositions are formulated for intravenous injection. Typically the compositions will be formulated in sterile saline or buffered solution for injection into the tissues or cells to be treated. The compositions can be stored lyophilized in single use vials for rehydration immediately before use. Other means for rehydration and administration are known to those skilled in the art.

Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions.

Generally, pharmaceutically acceptable salts can be prepared by reaction of the free acid or base forms of an agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, Md., 2000, p. 704. Examples of ophthalmic drugs sometimes administered in the form of a pharmaceutically acceptable salt include timolol maleate, brimonidine tartrate, and sodium diclofenac.

The compositions are preferably formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rabbits, dogs, or pigs. The animal model is also used to achieve a desirable concentration range and route of administration. Such information should then be useful to determine useful doses and routes for administration in humans. Therapeutic efficacy and toxicity of conjugates can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index and it can be expressed as the ratio, LD50/ED50. Pharmaceutical compositions which exhibit large therapeutic indices are preferred. The data obtained from cell culture assays and animal studies can be used in formulating a range of dosages for human use.

In certain embodiments, the compositions are administered locally, for example, by injection directly into a site to be treated. In some embodiments, the compositions are injected, topically applied, or otherwise administered directly into the vasculature onto vascular or mucosal tissue at or adjacent to a site of injury, surgery, or implantation. For example, in embodiments, the compositions are topically applied to vascular tissue that is exposed, during a surgical procedure. Typically, local administration causes an increased localized concentration of the compositions, which is greater than that which can be achieved by systemic administration.

Pharmaceutical compositions formulated for administration by parenteral (intramuscular, intraperitoneal, intravenous (IV) or subcutaneous injection) and enteral routes of administration are described.

A. Parenteral Administration

The phrases “parenteral administration” and “administered parenterally” are art-recognized terms, and include modes of administration other than enteral and topical administration, such as injections, and include without limitation intravenous (i.v.), intramuscular (i.m.), intrapleural, intravascular, intrapericardial, intraarterial, intrathecal, intracapsular, intraorbital, intracardiac, intradermal, intraperitoneal (i.p.), transtracheal, subcutaneous (s.c.), subcuticular, intraarticular, subcapsular, subarachnoid, intraspinal and intrasternal injection and infusion. The dendrimers can be administered parenterally, for example, by subdural, intravenous, intrathecal, intraventricular, intraarterial, intra-amniotic, intraperitoneal, or subcutaneous routes.

For liquid formulations, pharmaceutically acceptable carriers may be, for example, aqueous or non-aqueous solutions, suspensions, emulsions or oils. Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. The dendrimers can also be administered in an emulsion, for example, water in oil. Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer's dextrose. In general, water, saline, aqueous dextrose and related sugar solutions, and glycols such as propylene glycols or polyethylene glycol are preferred liquid carriers, particularly for injectable solutions.

Injectable pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Trissel, 15th ed., pages 622-630 (2009)).

B. Enteral Administration

The compositions can be administered enterally. The carriers or diluents may be solid carriers such as capsule or tablets or diluents for solid formulations, liquid carriers or diluents for liquid formulations, or mixtures thereof.

For liquid formulations, pharmaceutically acceptable carriers may be, for example, aqueous or non-aqueous solutions, suspensions, emulsions or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Vehicles include, for example, sodium chloride solution, Ringer's dextrose, dextrose and sodium chloride, lactated Ringer's and fixed oils. Formulations include, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Vehicles can include, for example, fluid and nutrient replenishers, electrolyte replenishers such as those based on Ringer's dextrose. In general, water, saline, aqueous dextrose and related sugar solutions are preferred liquid carriers. These can also be formulated with proteins, fats, saccharides and other components of infant formulas.

In some preferred embodiments, the compositions are formulated for oral administration. Oral formulations may be in the form of chewing gum, gel strips, tablets, capsules or lozenges. Encapsulating substances for the preparation of enteric-coated oral formulations include cellulose acetate phthalate, polyvinyl acetate phthalate, hydroxypropyl methylcellulose phthalate and methacrylic acid ester copolymers. Solid oral formulations such as capsules or tablets are preferred. Elixirs and syrups also are well known oral formulations.

### IV. Methods of Making Dendrimers and Conjugates or Complexes Thereof

A. Methods of Making Dendrimers

Dendrimers can be prepared via a variety of chemical reaction steps. Dendrimers are usually synthesized according to methods allowing controlling their structure at every stage of construction. The dendritic structures are mostly synthesized by two main different approaches: divergent or convergent.

In some embodiments, dendrimers are prepared using divergent methods, in which the dendrimer is assembled from a multifunctional core, which is extended outward by a series of reactions, commonly a Michael reaction. The strategy involves the coupling of monomeric molecules that possesses reactive and protective groups with the multifunctional core moiety, which leads to stepwise addition of generations around the core followed by removal of protecting groups. For example, PAMAM-NH₂ dendrimers are first synthesized by coupling N-(2-aminoethyl) acryl amide monomers to an ammonia core.

In other embodiments, dendrimers are prepared using convergent methods, in which dendrimers are built from small molecules that end up at the surface of the sphere, and reactions proceed inward, building inward, and are eventually attached to a core.

Many other synthetic pathways exist for the preparation of dendrimers, such as the orthogonal approach, accelerated approaches, the Double-stage convergent method or the hypercore approach, the hypermonomer method or the branched monomer approach, the Double exponential method; the Orthogonal coupling method or the two-step approach, the two monomers approach, AB₂-CD₂ approach.

In some embodiments, the core of the dendrimer, one or more branching units, one or more linkers/spacers, and/or one or more surface groups can be modified to allow conjugation to further functional groups (branching units, linkers/spacers, surface groups, etc.), monomers, and/or agents via click chemistry, employing one or more Copper-Assisted Azide-Alkyne Cycloaddition (CuAAC), Diels-Alder reaction, thiol-ene and thiol-yne reactions, and azide-alkyne reactions (Arseneault M et al., Molecules. 2015 May 20; 20(5):9263-94). In some embodiments, pre-made dendrons are clicked onto high-density hydroxyl polymers. ‘Click chemistry’ involves, for example, the coupling of two different moieties (e.g., a core group and a branching unit; or a branching unit and a surface group) via a 1,3-dipolar cycloaddition reaction between an alkyne moiety (or equivalent thereof) on the surface of the first moiety and an azide moiety (e.g., present on a triazine composition or equivalent thereof), or any active end group such as, for example, a primary amine end group, a hydroxyl end group, a carboxylic acid end group, a thiol end group, etc.) on the second moiety.

In some embodiments, dendrimer synthesis replies upon one or more reactions such as thiol-ene click reactions, thiol-yne click reactions, CuAAC, Diels-Alder click reactions, azide-alkyne click reactions, Michael Addition, epoxy opening, esterification, silane chemistry, and a combination thereof.

Any existing dendritic platforms can be used to make dendrimers of desired functionalities, i.e., with a high-density of surface hydroxyl groups by conjugating high-hydroxyl containing moieties such as 1-thio-glycerol or pentaerythritol. Exemplary dendritic platforms such as polyamidoamine (PAMAM), poly (propylene imine) (PPI), poly-L-lysine, melamine, poly (etherhydroxylamine) (PEHAM), poly (esteramine) (PEA) and polyglycerol can be synthesized and explored.

Dendrimers also can be prepared by combining two or more dendrons. Dendrons are wedge-shaped sections of dendrimers with reactive focal point functional groups. Many dendron scaffolds are commercially available. They come in 1, 2, 3, 4, 5, and 6th generations with, respectively, 2, 4, 8, 16, 32, and 64 reactive groups. In certain embodiments, one type of agents are linked to one type of dendron and a different type of agent is linked to another type of dendron. The two dendrons are then connected to form a dendrimer. The two dendrons can be linked via click chemistry i.e., a 1,3-dipolar cycloaddition reaction between an azide moiety on one dendron and alkyne moiety on another to form a triazole linker.

Exemplary methods of making dendrimers are described in detail in International Patent Publication Nos. WO2009/046446, WO2015168347, WO2016025745, WO2016025741, WO2019094952, and U.S. Pat. No. 8,889,101.

B. Dendrimer Complexes

Dendrimer complexes can be formed of therapeutic, prophylactic or diagnostic agents conjugated or complexed to a dendrimer, a dendritic polymer or a hyperbranched polymer. Conjugation of one or more agents to a dendrimer are known in the art, and are described in detail in U.S. Published Application Nos. US 2011/0034422, US 2012/0003155, and US 2013/0136697.

In some embodiments, one or more agents are covalently attached to the dendrimers. In some embodiments, the agents are attached to the dendrimer via a linking moiety that is designed to be cleaved in vivo. The linking moiety can be designed to be cleaved hydrolytically, enzymatically, or combinations thereof, so as to provide for the sustained release of the agents in vivo. Both the composition of the linking moiety and its point of attachment to the agent, are selected so that cleavage of the linking moiety releases either an agent, or a suitable prodrug thereof. The composition of the linking moiety can also be selected in view of the desired release rate of the agents.

In some embodiments, the attachment occurs via one or more of disulfide, ester, ether, thioester, carbamate, carbonate, hydrazine, or amide linkages. In preferred embodiments, the attachment occurs via an appropriate spacer that provides an ester bond or an amide bond between the agent and the dendrimer depending on the desired release kinetics of the agent. In some cases, an ester bond is introduced for releasable form of agents. In other cases, an amide bond is introduced for non-releasable form of agents.

Linking moieties generally include one or more organic functional groups. Examples of suitable organic functional groups include secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, hydrazones, hydrazides, ethers (—O—), and esters (—COO—, —CH₂O₂C—, CHRO₂C—), wherein R is an alkyl group, an aryl group, or a heterocyclic group. In general, the identity of the one or more organic functional groups within the linking moiety is chosen in view of the desired release rate of the agents. In addition, the one or more organic functional groups can be selected to facilitate the covalent attachment of the agents to the dendrimers. In preferred embodiments, the attachment can occur via an appropriate spacer that provides a disulfide bridge between the agent and the dendrimer. The dendrimer complexes are capable of rapid release of the agent in vivo by thiol exchange reactions, under the reduced conditions found in body.

In certain embodiments, the linking moiety includes one or more of the organic functional groups described above in combination with a spacer group. The spacer group can be composed of any assembly of atoms, including oligomeric and polymeric chains; however, the total number of atoms in the spacer group is preferably between 3 and 200 atoms, more preferably between 3 and 150 atoms, more preferably between 3 and 100 atoms, most preferably between 3 and 50 atoms. Examples of suitable spacer groups include alkyl groups, heteroalkyl groups, alkylaryl groups, oligo- and polyethylene glycol chains, and oligo- and poly(amino acid) chains. Variation of the spacer group provides additional control over the release of the agents in vivo. In embodiments where the linking moiety includes a spacer group, one or more organic functional groups will generally be used to connect the spacer group to both the anti-inflammatory agent and the dendrimers.

Reactions and strategies useful for the covalent attachment of agents to dendrimers are known in the art. See, for example, March, “Advanced Organic Chemistry,” 5th Edition, 2001, Wiley-Interscience Publication, New York) and Hermanson, “Bioconjugate Techniques,” 1996, Elsevier Academic Press, U.S.A. Appropriate methods for the covalent attachment of a given agent can be selected in view of the linking moiety desired, as well as the structure of the agents and dendrimers as a whole as it relates to compatibility of functional groups, protecting group strategies, and the presence of labile bonds.

The optimal drug loading will necessarily depend on many factors, including the choice of drug, dendrimer structure and size, and tissues to be treated. In some embodiments, the one or more agents are encapsulated, associated, and/or conjugated to the dendrimer at a concentration of about 0.01% to about 45%, preferably about 0.1% to about 30%, about 0.1% to about 20%, about 0.1% to about 10%, about 1% to about 10%, about 1% to about 5%, about 3% to about 20% by weight, and about 3% to about 10% by weight. However, optimal drug loading for any given drug, dendrimer, and site of target can be identified by routine methods, such as those described.

In some embodiments, conjugation of agents and/or linkers occurs through one or more surface and/or interior groups. Thus, in some embodiments, the conjugation of agents/linkers occurs via about 1%, 2%, 3%, 4%, or 5% of the total available surface functional groups, preferably hydroxyl groups, of the dendrimers prior to the conjugation. In other embodiments, the conjugation of agents/linkers occurs on less than 5%, less than 10%, less than 15%, less than 20%, less than 25%, less than 30%, less than 35%, less than 40%, less than 45%, less than 50%, less than 55%, less than 60%, less than 65%, less than 70%, less than 75% total available surface functional groups of the dendrimers prior to the conjugation. In preferred embodiments, dendrimer complexes retain an effective amount of surface functional groups for targeting to specific cell types, whilst conjugated to an effective amount of agents for treat, prevent, and/or image the disease or disorder.

### V. Methods of Use

Methods of using the dendrimer complex compositions are also described. In preferred embodiments, the dendrimer complexes cross impaired or damaged BBB and target activated microglia and astrocytes. The methods can be used for treating one or more conditions and/or diseases associated with elevated levels and/or activities of neutral sphingomyelinase 2 (nSMase2). Methods can also be used for treating one or more conditions and/or diseases associated with elevated levels and/or activities of ceramide are also provided. In some embodiments, the methods are used to effectively reduce exosome biosynthesis. The methods include administering an effective amount of a composition including dendrimer complexed with, conjugated to, or encapsulated with one or more inhibitors of nSMase2 to a subject in need thereof. In preferred embodiments, the methods include administering an effective amount of a composition including dendrimer complexed with or conjugated to 2,6-dimethoxy-4-(5-phenyl-4-(thiophen-2-yl)-1H-imidazol -2-yl) phenol (DPTIP), or a derivative or analog, or pharmaceutically acceptable salt thereof to the subject.

A. Methods of Treatment

The dendrimer compositions and formulations thereof can be administered to treat disorders associated with infection, inflammation, or cancer, particularly those having systemic inflammation that extends to the nervous system, especially the CNS. The compositions can also be used for treatment of other diseases, disorders and injury including gastrointestinal disorders, proliferative diseases and treatment of other tissues where the nerves play a role in the disease or disorder. The compositions and methods are also suitable for prophylactic use.

Typically, an effective amount of dendrimer complexes including a combination of a dendrimer with one or more therapeutic, prophylactic, and/or diagnostic active agents are administered to an individual in need thereof. The dendrimers may also include a targeting agent, but as demonstrated by the examples, these are not required for delivery to injured tissue in the spinal cord and the brain.

In some embodiments, the dendrimer complexes include an agent(s) that is attached or conjugated to dendrimers, which are capable of preferentially releasing the drug intracellularly under the reduced conditions found in vivo. The agent can be either covalently attached or intra-molecularly dispersed or encapsulated. The amount of dendrimer complexes administered to the subject is selected to deliver an effective amount to reduce, prevent, or otherwise alleviate one or more clinical or molecular symptoms of the disease or disorder to be treated compared to a control, for example, a subject treated with the active agent without dendrimer.

B. Conditions to be Treated

The compositions are suitable for treating one or more diseases, conditions, and injuries in the eye, the brain, and the nervous system, particularly those associated with pathological activation of microglia and astrocytes, cancer, infectious disease, and inflammatory disorders.

Microglia are a type of neuroglia (glial cell) located throughout the brain and spinal cord. Microglia account for 10-15% of all cells found within the brain. As the resident macrophage cells, they act as the first and main form of active immune defense in the central nervous system (CNS). Microglia play a key role after CNS injury, and can have both protective and deleterious effects based on the timing and type of insult (Kreutzberg, G. W. Trends in Neurosciences, 19, 312 (1996); Watanabe, H., et al., Neuroscience Letters, 289, 53 (2000); Polazzi, E., et al., Glia, 36, 271 (2001); Mallard, C., et al., Pediatric Research, 75, 234 (2014); Faustino, J. V., et al., The Journal of Neuroscience: The Official Journal Of The Society For Neuroscience, 31, 12992 (2011); Tabas, I., et al., Science, 339, 166 (2013); and Aguzzi, A., et al., Science, 339, 156 (2013)). Changes in microglial function also affect normal neuronal development and synaptic pruning (Lawson, L. J., et al., Neuroscience, 39, 151 (1990); Giulian, D., et al., The Journal Of Neuroscience: The Official Journal Of The Society For Neuroscience, 13, 29 (1993); Cunningham, T. J., et al., The Journal of Neuroscience: The Official Journal Of The Society For Neuroscience, 18, 7047 (1998); Zietlow, R., et al., The European Journal Of Neuroscience, 11, 1657 (1999); and Paolicelli, R. C., et al., Science, 333, 1456 (2011)). Microglia undergo a pronounced change in morphology from ramified to an amoeboid structure and proliferate after injury. The resulting neuroinflammation disrupts the blood-brain-barrier at the injured site, and cause acute and chronic neuronal and oligodendrocyte death. Hence, targeting pro-inflammatory microglia should be a potent and effective therapeutic strategy. The impaired BBB in neuroinflammatory diseases can be exploited for transport of drug carrying nanoparticles into the brain.

1. Neurological and Neurodegenerative Diseases

The dendrimer compositions and formulations thereof can be used to diagnose and/or to treat one or more neurological and neurodegenerative diseases. The compositions and methods are particularly suited for treating one or more neurological, or neurodegenerative diseases associated with defective metabolism and functions of sphingolipids including sphingomyelin. In some embodiments, the disease or disorder is selected from, but not limited to, some psychiatric (e.g., depression, schizophrenia (SZ), alcohol use disorder, and morphine antinociceptive tolerance) and neurological (e.g., Alzheimer's disease (AD), Parkinson disease (PD)) disorders. In one embodiment, the dendrimer complexes are used to treat Alzheimer's Disease (AD) or dementia.

Neurodegenerative diseases are chronic progressive disorders of the nervous system that affect neurological and behavioral function and involve biochemical changes leading to distinct histopathologic and clinical syndromes (Hardy H, et al., Science. 1998; 282:1075-9). Abnormal proteins resistant to cellular degradation mechanisms accumulate within the cells. The pattern of neuronal loss is selective in the sense that one group gets affected, whereas others remain intact. Often, there is no clear inciting event for the disease. The diseases classically described as neurodegenerative are Alzheimer's disease, Huntington's disease, and Parkinson's disease.

Neuroinflammation, mediated by activated microglia and astrocytes, is a major hallmark of various neurological disorders making it a potential therapeutic target (Hagberg, H et al., Annals of Neurology 2012, 71, 444; Vargas, D L et al., Annals of Neurology 2005, 57, 67; and Pardo, C A et al., International Review of Psychiatry 2005, 17, 485). Multiple scientific reports suggest that mitigating neuroinflammation in early phase by targeting these cells can delay the onset of disease and can in turn provide a longer therapeutic window for the treatment (Dommergues, M A et al., Neuroscience 2003, 121, 619; Perry, V H et al., Nat Rev Neurol 2010, 6, 193; Kannan, S et al., Sci. Transl. Med. 2012, 4, 130ra46; and Block, M L et al., Nat Rev Neurosci 2007, 8, 57). The delivery of therapeutics across blood brain barrier is a challenging task. The neuroinflammation causes disruption of blood brain barrier (BBB). The impaired BBB in neuroinflammatory disorders can be utilized to transport drug loaded nanoparticles across the brain (Stolp, H B et al., Cardiovascular Psychiatry and Neurology 2011, 2011, 10; and Ahishali, B et al., International Journal of Neuroscience 2005, 115, 151).

The compositions and methods can also be used to deliver active agents for the treatment of a neurological or neurodegenerative disease or disorder or central nervous system disorder. In preferred embodiments, the compositions and methods are effective in treating, and/or alleviating neuroinflammation associated with a neurological or neurodegenerative disease or disorder or central nervous system disorder. The methods typically include administering to the subject an effective amount of the composition to increase cognition or reduce a decline in cognition, increase a cognitive function or reduce a decline in a cognitive function, increase memory or reduce a decline in memory, increase the ability or capacity to learn or reduce a decline in the ability or capacity to learn, or a combination thereof.

Neurodegeneration refers to the progressive loss of structure or function of neurons, including death of neurons. For example, the compositions and methods can be used to treat subjects with a disease or disorder, such as Parkinson's Disease (PD) and PD-related disorders, Huntington's Disease (HD), Amyotrophic Lateral Sclerosis (ALS), Alzheimer's Disease (AD) and other dementias, Prion Diseases such as Creutzfeldt-Jakob Disease, Corticobasal Degeneration, Frontotemporal Dementia, HIV-Related Cognitive Impairment, Mild Cognitive Impairment, Motor Neuron Diseases (MND), Spinocerebellar Ataxia (SCA), Spinal Muscular Atrophy (SMA), Friedreich's Ataxia, Lewy Body Disease, Alpers' Disease, Batten Disease, Cerebro-Oculo-Facio-Skeletal Syndrome, Corticobasal Degeneration, Gerstmann-Straussler-Scheinker Disease, Kuru, Leigh's Disease, Monomelic Amyotrophy, Multiple System Atrophy, Multiple System Atrophy With Orthostatic Hypotension (Shy-Drager Syndrome), Multiple Sclerosis (MS), Duchenne muscular dystrophy (DMD), Neurodegeneration with Brain Iron Accumulation, Opsoclonus Myoclonus, Posterior Cortical Atrophy, Primary Progressive Aphasia, Progressive Supranuclear Palsy, Vascular Dementia, Progressive Multifocal Leukoencephalopathy, Dementia with Lewy Bodies (DLB), Lacunar syndromes, Hydrocephalus, Wernicke-Korsakoff's syndrome, post-encephalitic dementia, cancer and chemotherapy-associated cognitive impairment and dementia, and depression-induced dementia and pseudodementia.

In further embodiments, the disease or disorder is selected from, but not limited to, injection-localized amyloidosis, cerebral amyloid angiopathy, myopathy, neuropathy, brain trauma, frontotemporal dementia, Pick's disease, multiple sclerosis, prion disorders, diabetes mellitus type 2, fatal familial insomnia, cardiac arrhythmias, isolated atrial amyloidosis, atherosclerosis, rheumatoid arthritis, familial amyloid polyneuropathy, hereditary non-neuropathic systemic amyloidosis, Finnish amyloidosis, lattice corneal dystrophy, systemic AL amyloidosis, and Down syndrome. In preferred embodiments, the disease or disorder is Alzheimer's disease or dementia.

Criteria for assessing improvement in a particular neurological factor include methods of evaluating cognitive skills, motor skills, memory capacity or the like, as well as methods for assessing physical changes in selected areas of the central nervous system, such as magnetic resonance imaging (MRI) and computed tomography scans (CT) or other imaging methods. Such methods of evaluation are well known in the fields of medicine, neurology, psychology and the like, and can be appropriately selected to diagnosis the status of a particular neurological impairment. To assess a change in Alzheimer's disease, or related neurological changes, the selected assessment or evaluation test, or tests, are given prior to the start of administration of the dendrimer compositions. Following this initial assessment, treatment methods for the administration of the dendrimer compositions are initiated and continued for various time intervals. At a selected time interval subsequent to the initial assessment of the neurological defect impairment, the same assessment or evaluation test (s) is again used to reassess changes or improvements in selected neurological criteria.

a. Alzheimer's Disease and Dementia

Brains from Alzheimer's disease (AD) patients show elevated ceramide, an integral component of exosomal membranes. One major source of ceramide is through the hydrolysis of sphingomyelin catalyzed by neutral sphingomyelinase 2 (nSMase2). Recent studies show that chronically activated nSMase2 is implicated in both Ab aggregation and tau propagation through its role in exosome secretion.

The dendrimer compositions are suitable for reducing or preventing one or more pathological processes associated with the development and progression of neurological diseases such as Alzheimer's disease and dementia. Thus, methods for treatment, reduction, and prevention of the pathological processes associated with Alzheimer's disease include administering the dendrimer compositions in an amount and dosing regimen effective to reduce brain and/or serum exosomes, brain and/or serum ceramide levels, serum anti-ceramide IgG, glial activation, total Aβ42 and plaque burden, tau phosphorylation/propagation, and improved cognition in a learning task, such as a fear-conditioned learning task, in an individual suffering from Alzheimer's disease or dementia are provided. Methods for reducing, preventing, or reversing the learning and/or memory deficits in an individual suffering from Alzheimer's disease or dementia are provided. The methods include administering an effective amount of a composition including dendrimer complexed with, conjugated to, or encapsulated with one or more inhibitors of sphingomyelinase to a subject in need thereof. In preferred embodiments, the methods include administering an effective amount of a composition including dendrimer complexed with or conjugated to 2,6-dimethoxy-4-(5-phenyl-4-(thiophen-2-yl)-1H-imidazol -2-yl) phenol (DPTIP), or a derivative or analog, or pharmaceutically acceptable salt thereof to the subject.

In some embodiments, the dendrimer compositions are administered in an amount and dosing regimen effective to induce neuro-enhancement in a subject in need thereof. Neuro-enhancement resulting from the administration of the dendrimer compositions includes the stimulation or induction of neural mitosis leading to the generation of new neurons, i.e., exhibiting a neurogenic effect, prevention or retardation of neural loss, including a decrease in the rate of neural loss, i.e., exhibiting a neuroprotective effect, or one or more of these modes of action. The term “neuroprotective effect” is intended to include prevention, retardation, and/or termination of deterioration, impairment, or death of an individual's neurons, neurites and neural networks. Administration of the compositions leads to an improvement, or enhancement, of neurological function in an individual with a neurological disease, neurological injury, or age-related neuronal decline or impairment.

Neural deterioration can be the result of any condition which compromises neural function which is likely to lead to neural loss. Neural function can be compromised by, for example, altered biochemistry, physiology, or anatomy of a neuron, including its neurite. Deterioration of a neuron may include membrane, dendritic, or synaptic changes, which are detrimental to normal neuronal functioning. The cause of the neuron deterioration, impairment, and/or death may be unknown. Alternatively, it may be the result of age-, injury-and/or disease-related neurological changes that occur in the nervous system of an individual.

In Alzheimer's patients, neural loss is most notable in the hippocampus, frontal, parietal, and anterior temporal cortices, amygdala, and the olfactory system. The most prominently affected zones of the hippocampus include the CA1 region, the subiculum, and the entorhinal cortex. Memory loss is considered the earliest and most representative cognitive change because the hippocampus is well known to play a crucial role in memory.

Neural loss through disease, age-related decline or physical insult leads to neurological disease and impairment. The compositions can counteract the deleterious effects of neural loss by promoting development of new neurons, new neurites and/or neural connections, resulting in the neuroprotection of existing neural cells, neurites and/or neural connections, or one or more these processes. Thus, the neuro-enhancing properties of the compositions provide an effective strategy to generally reverse the neural loss associated with degenerative diseases, aging and physical injury or trauma.

Administration of the dendrimer compositions to an individual who is undergoing or has undergone neural loss, as a result of Alzheimer's disease reduces any one or more of the symptoms of Alzheimer's disease, or associated cognitive disorders, including dementia. Clinical symptoms of AD or dementia that can be treated, reduced or prevented include clinical symptoms of mild AD, moderate AD, and/or severe AD or dementia.

In mild Alzheimer's disease, a person may seem to be healthy but has more and more trouble making sense of the world around him or her. The realization that something is wrong often comes gradually to the person and their family Exemplary symptoms of mild Alzheimer's disease/mild dementia include, but are not limited to, memory loss; poor judgment leading to bad decisions; loss of spontaneity and sense of initiative; taking longer to complete normal daily tasks; repeating questions; trouble handling money and paying bills; wandering and getting lost; losing things or misplacing them in odd places; mood and personality changes, and increased anxiety and/or aggression.

Symptoms of moderate Alzheimer's disease/moderate dementia include, but are not limited to forgetfulness; increased memory loss and confusion; inability to learn new things; difficulty with language and problems with reading, writing, and working with numbers; difficulty organizing thoughts and thinking logically; shortened attention span; problems coping with new situations; difficulty carrying out multistep tasks, such as getting dressed; problems recognizing family and friends; hallucinations, delusions, and paranoia; impulsive behavior such as undressing at inappropriate times or places or using vulgar language; inappropriate outbursts of anger; restlessness, agitation, anxiety, tearfulness, wandering (especially in the late afternoon or evening); repetitive statements or movement, occasional muscle twitches.

Symptoms of severe Alzheimer's disease/severe dementia include, but are not limited to inability to communicate; weight loss; seizures; skin infections; difficulty swallowing; groaning, moaning, or grunting; increased sleeping; loss of bowel and bladder control.

Physiological symptoms of Alzheimer's disease/dementia include reduction in brain mass, for example, reduction in hippocampal volume. Therefore, in some embodiments, methods of administering the disclosed compositions increase the brain mass, and/or reduce or prevent the rate of decrease in brain mass of a subject; increase the hippocampal volume of the subject, reduce or prevent the rate of decrease of hippocampal volume, as compared to an untreated control subject.

The compositions are administered in an amount that is effective to reduce brain exosomes, ceramide levels, serum anticeramide IgG, glial activation, total Aβ₄₂ and plaque burden, tau phosphorylation, improved cognition in a fear-conditioned learning task, and combinations thereof.

The dendrimer compositions are administered to provide an effective amount of one or more therapeutic agents (e.g., inhibitors of sphingomyelinase) upon administration to an individual. As used in this context, an “effective amount” of one or more therapeutic agents is an amount that is effective to improve or ameliorate one or more symptoms associated with Alzheimer's disease or dementia, including neurological defects or cognitive decline or impairment. Such a therapeutic effect is generally observed within about 12 to about 24 weeks of initiating administration of a composition containing an effective amount of one or more neuro-enhancing agents, although the therapeutic effect may be observed in less than 12 weeks or greater than 24 weeks.

The individual is preferably an adult human, and more preferably, a human is over the age of 30, who has lost some amount of neurological function as a result of Alzheimer's disease or dementia. Generally, neural loss implies any neural loss at the cellular level, including loss in neurites, neural organization or neural networks.

In other embodiments, the methods including selecting a subject who is likely to benefit from treatment with the dendrimer compositions. For example, ceramide levels in the CSF of a patient is first determined and compared to that of a healthy control. In some embodiments, the dendrimer compositions are administered to a patient having an elevated concentration of ceramide in the CSF or in the serum relative to that of a healthy control. In other embodiments, the dendrimer compositions are administered to a patient with increased quantity of brain and/or serum exosomes relative to that of a healthy control. In other embodiments, the dendrimer compositions are administered to a patient with increased levels of serum anti-ceramide IgG relative to that of a healthy control. In other embodiments, the dendrimer compositions are administered to a patient with altered or aberrant metabolic activities involving one or more enzymatic or receptor-mediated mechanisms in microglia such as nSMase2, TREM2, LRRK2, and RIPK1.

2. Cancer

In some embodiments, the dendrimer compositions and formulations thereof are used in a method for treating a cancer in a subject in need of. The method for treating a cancer in a subject in need of including administering to the subject a therapeutically effective amount of the dendrimer compositions.

In preferred cases, the dendrimer compositions and formulations thereof are administered in an amount effective to inhibit tumor growth, reduce tumor size, increase rates of long-term survival, improve response to immune checkpoint blockade, and/or induce immunological memory that protects against tumor re-challenge.

A cancer in a patient refers to the presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells will be in the form of a tumor; such cells may exist locally within an animal, or circulate in the blood stream as independent cells, for example, leukemic cells. A tumor refers to all neoplastic cell growth and proliferation, whether malignant or benign, and all precancerous and cancerous cells and tissues. A solid tumor is an abnormal mass of tissue that generally does not contain cysts or liquid areas. A solid tumor may be in the brain, colon, breasts, prostate, liver, kidneys, lungs, esophagus, head and neck, ovaries, cervix, stomach, colon, rectum, bladder, uterus, testes, and pancreas, as non-limiting examples. In some embodiments, the solid tumor regresses or its growth is slowed or arrested after the solid tumor is treated with the presently disclosed methods. In other embodiments, the solid tumor is malignant. In some embodiments, the cancer comprises Stage 0 cancer. In some embodiments, the cancer comprises Stage I cancer. In some embodiments, the cancer comprises Stage II cancer. In some embodiments, the cancer comprises Stage III cancer. In some embodiments, the cancer comprises Stage IV cancer. In some embodiments, the cancer is refractory and/or metastatic. For example, the cancer may be refractory to treatment with radiotherapy, chemotherapy or monotreatment with immunotherapy. Cancer includes newly diagnosed or recurrent cancers, including without limitation, acute lymphoblastic leukemia, acute myelogenous leukemia, advanced soft tissue sarcoma, brain cancer, metastatic or aggressive breast cancer, breast carcinoma, bronchogenic carcinoma, choriocarcinoma, chronic myelocytic leukemia, colon carcinoma, colorectal carcinoma, Ewing's sarcoma, gastrointestinal tract carcinoma, glioma, glioblastoma multiforme, head and neck squamous cell carcinoma, hepatocellular carcinoma, Hodgkin's disease, intracranial ependymoblastoma, large bowel cancer, leukemia, liver cancer, lung carcinoma, Lewis lung carcinoma, lymphoma, malignant fibrous histiocytoma, a mammary tumor, melanoma, mesothelioma, neuroblastoma, osteosarcoma, ovarian cancer, pancreatic cancer, a pontine tumor, premenopausal breast cancer, prostate cancer, rhabdomyosarcoma, reticulum cell sarcoma, sarcoma, small cell lung cancer, a solid tumor, stomach cancer, testicular cancer, and uterine carcinoma. In some embodiments, the cancer is acute leukemia. In some embodiments, the cancer is acute lymphoblastic leukemia. In some embodiments, the cancer is acute myelogenous leukemia. In some embodiments, the cancer is advanced soft tissue sarcoma. In some embodiments, the cancer is a brain cancer. In some embodiments, the cancer is breast cancer (e.g., metastatic or aggressive breast cancer). In some embodiments, the cancer is breast carcinoma. In some embodiments, the cancer is bronchogenic carcinoma. In some embodiments, the cancer is choriocarcinoma. In some embodiments, the cancer is chronic myelocytic leukemia. In some embodiments, the cancer is a colon carcinoma (e.g., adenocarcinoma). In some embodiments, the cancer is colorectal cancer (e.g., colorectal carcinoma). In some embodiments, the cancer is Ewing's sarcoma. In some embodiments, the cancer is gastrointestinal tract carcinoma. In some embodiments, the cancer is a glioma. In some embodiments, the cancer is glioblastoma multiforme. In some embodiments, the cancer is head and neck squamous cell carcinoma. In some embodiments, the cancer is hepatocellular carcinoma. In some embodiments, the cancer is Hodgkin's disease. In some embodiments, the cancer is intracranial ependymoblastoma. In some embodiments, the cancer is large bowel cancer. In some embodiments, the cancer is leukemia. In some embodiments, the cancer is liver cancer. In some embodiments, the cancer is lung cancer (e.g., lung carcinoma). In some embodiments, the cancer is Lewis lung carcinoma. In some embodiments, the cancer is lymphoma. In some embodiments, the cancer is malignant fibrous histiocytoma. In some embodiments, the cancer comprises a mammary tumor. In some embodiments, the cancer is melanoma. In some embodiments, the cancer is mesothelioma. In some embodiments, the cancer is neuroblastoma. In some embodiments, the cancer is osteosarcoma. In some embodiments, the cancer is ovarian cancer. In some embodiments, the cancer is pancreatic cancer. In some embodiments, the cancer comprises a pontine tumor. In some embodiments, the cancer is premenopausal breast cancer. In some embodiments, the cancer is prostate cancer. In some embodiments, the cancer is rhabdomyosarcoma. In some embodiments, the cancer is reticulum cell sarcoma. In some embodiments, the cancer is sarcoma. In some embodiments, the cancer is small cell lung cancer. In some embodiments, the cancer comprises a solid tumor. In some embodiments, the cancer is stomach cancer. In some embodiments, the cancer is testicular cancer. In some embodiments, the cancer is uterine carcinoma. In some embodiments, the cancer is multiple myeloma. In some embodiments, the cancer is skin cancer. In some embodiments, the cancer is duodenal cancer.

3. Cardiac Disease

In some embodiments, the dendrimer compositions and formulations thereof are used in a method for treating cardiac disease in a subject in need of. The method for treating cardiac disease including administering to the subject a therapeutically effective amount of the dendrimer compositions. In particular embodiments, the cardiac disease is a myocardial disease involving myocyte hypertrophy, fibroblast-derived cardiac hypertrophy, heart failure, heart hypertrophy, diastolic and/or systolic ventricular dysfunction and/or a cardiovascular disease involving fibrosis, aortic stenosis, atrial fibrillation, genetic forms of cardiomyopathy, cardiac storage diseases and/or fabry disease.

4. Infectious Disease

The formulations are effective in treating disease resulting from viral, bacterial, parasitic and fungal infections, or inflammation associated therewith.

Exemplary infection-causing agents include human immunodeficiency virus (HIV), Zika virus, Hepatitis C, Hepatitis E, Rabies, Langat virus (LGTV), Dengue virus (DENV), cytomegalovirus (HCMV), and Newcastle disease virus (NDV), Epsilon-toxin from Clostridium perfringens, and shiga toxin from Escherichia coli. For example, EVs are implicated in the propagation of human immunodeficiency virus (HIV) infection (reviewed in Caobi, A. et al., Viruses 12 (10), 1200 (2020)). EVs released from HIV-infected cells carry HIV accessory proteins and co-receptors that make target cells more receptive to HIV infection. Additionally, the virion can physically associate with EVs which can enable it to evade immune surveillance and increase infectivity. It has also been shown that EVs from HIV-1-infected CD4+ T cells can induce HIV-1 reactivation from dormant viral reservoirs in resting CD4+ T lymphocytes (Chiozzini, C. et al. Archives of Virology 162 (9), 2565-2577 (2017)). In cell culture experiments, blocking the release of EVs from infected CD4+ T cells with the nSMase2 inhibitors GW4869 and spiroepoxide reduced dendritic cell-mediated infection of healthy CD4+ T lymphocytes.

In addition to HIV, nSMase2 inhibitors have also shown therapeutic promise against the Zika virus. Zika infection in human fetal astrocytes was shown to increase release of EVs and viral particles; some of the viral particles were packaged within EVs. Inhibiting EV release via GW4869 led to diminished Zika virus propagation (Huang, Y. et al., Cell discovery 4, 19-19 (2018)). Similar findings were observed in murine neuronal cell cultures where Zika virus led to enhanced EV release containing viral RNA. Either silencing nSMase2 using siRNA or pharmacologically inhibiting the enzyme with GW4869 reduced EV release and diminished viral RNA levels [101]. The efficacy of nSMase2 inhibitors has also been explored in Hepatitis C, Hepatitis E, Rabies, Langat virus (LGTV), Dengue virus (DENV), cytomegalovirus (HCMV), and Newcastle disease virus (NDV).

In some embodiments, the dendrimer compositions and formulations thereof are used for reducing or inhibiting viral replication, viral load, and/or viral release, particularly in cases where activated microglia and astrocytes are targeted/infected by the virus.

Epsilon-toxin produced by Clostridium perfringens, a lethal bacterial infection of undulates, was shown to enhance ceramide production in exposed kidney cells. Treatment of the exposed kidney cells with GW4869 reduced cell-death (Takagishi, T. et al., Biochimica et Biophysica Acta (BBA)—Biomembranes 1858 (11), 2681-2688 (2016)). The bacterial shiga toxin, released by certain strains of Escherichia coli which is associated with GI, kidney, and CNS pathology, was found to be packaged into EVs derived from exposed macrophages. These EVs induced cell death in naïve HK-2 renal epithelial cells. Renal epithelial cell death rates were ameliorated when EV release was blocked with nSMase2 inhibition (Lee, K.-S. et al., Cellular Microbiology n/a (n/a), e13249 (2020)).

Accordingly, the dendrimer compositions and formulations thereof are used in a method for treating one or more bacterial, parasitic, fungal or viral infections, or inflammation associated therewith.

5. Inflammatory Diseases

EVs have been shown to be involved in the inflammatory response to airway diseases. In a mouse model of allergic airway inflammation, treatment with the nSMase2 inhibitor GW4869 led to fewer lung macrophages and improved airway hyper-responsiveness and bronchial pathology (Kulshreshtha, A. et al., Journal of Allergy and Clinical Immunology 131 (4), 1194-1203.e1114 (2013)).

Inhibiting nSMase2 can improve outcomes of ischemia-reperfusion (IR) injury. In preclinical cerebral ischemia models, blocking pro-inflammatory EV release from brain tissue with GW4869 resulted in fewer Iba1+ cells in the cortex and hippocampus and a shift in microglia from the pro-inflammatory state to anti-inflammatory state as measured by a decrease in CD86 and increase in CD206 levels and a reduction of inflammatory markers (Gao, G. et al., Frontiers in immunology 11, 161-161 (2020); Gu, L. et al., Journal of Neuroinflammation 10 (1), 879 (2013)).

Chronic endothelial inflammation is implicated is atherosclerosis. In hypertensive patients, endothelin-1 is elevated and activates nSMase2, which increases vascular cell adhesion protein 1 (VCAM-1) and vascular inflammation leading to small artery remodeling. Inhibiting nSMase2 with GW4869 lowers VCAM-1 expression in rat mesenteric small arteries (Ohanian, J. et al., Journal of Vascular Research 49 (4), 353-362 (2012)).

Accordingly, the dendrimer compositions and formulations thereof are used in a method for treating one or more inflammatory diseases. Exemplary inflammatory diseases include airway inflammation, allergic airway inflammation, atherosclerosis, cerebral ischemia, hepatic ischemia reperfusion injury, myocardial infarction, and sepsis.

C. Dosage and Effective Amounts

Dosage and dosing regimens are dependent on the severity and nature of the disorder or injury, as well as the route and timing of administration, and can be determined by those skilled in the art. A therapeutically effective amount of the dendrimer composition used in the treatment of a neurological or neurodegenerative disease is typically sufficient to reduce or alleviate one or more symptoms of the neurological or neurodegenerative disease, or to reduce inflammation or severity of disease in other conditions.

Preferably, the agents do not target or otherwise modulate the activity or quantity of healthy cells not within or associated with the diseased or target tissues, or do so at a reduced level compared to target cells including activated microglial cells in the CNS. In this way, by-products and other side effects associated with the compositions are reduced.

Administration of the compositions leads to an improvement, or enhancement, of neurological function in an individual with a neurological disease, neurological injury, or age-related neuronal decline or impairment. In some in vivo approaches, the dendrimer complexes are administered to a subject in a therapeutically effective amount to stimulate or induce neural mitosis leading to the generation of new neurons, providing a neurogenic effect. Also provided are effective amounts of the compositions to prevent, reduce, or terminate deterioration, impairment, or death of an individual's neurons, neurites and neural networks, providing a neuroprotective effect.

The actual effective amounts of dendrimer complex can vary according to factors including the specific agent administered, the particular composition formulated, the mode of administration, and the age, weight, condition of the subject being treated, as well as the route of administration and the disease or disorder. The dose of the compositions can be from about 0.01 to about 100 mg/kg body weight, from about 0.1 mg/kg to about 50 mg/kg, from about 0.5 mg to about 40 mg/kg body weight, and from about 2 mg to about 10 mg/kg body weight. Generally, for intravenous injection or infusion, the dosage may be lower than for oral administration.

In general, the timing and frequency of administration will be adjusted to balance the efficacy of a given treatment or diagnostic schedule with the side-effects of the given delivery system. Exemplary dosing frequencies include continuous infusion, single and multiple administrations such as hourly, daily, weekly, or monthly dosing.

The compositions can be administered daily, biweekly, weekly, every two weeks or less frequently in an amount to provide a therapeutically effective increase in the blood level of the therapeutic agent. Where the administration is by other than an oral route, the compositions may be delivered over a period of more than one hour, e.g., 3-10 hours, to produce a therapeutically effective dose within a 24-hour period. Alternatively, the compositions can be formulated for controlled release, wherein the composition is administered as a single dose that is repeated on a regimen of once a week, or less frequently.

Dosage can vary, and can be administered in one or more dose administrations daily, for one or several days. Guidance can be found in the literature for appropriate dosages for given classes of pharmaceutical products. Optimal dosing schedules can be calculated from measurements of drug accumulation in the body of the subject or patient. Persons of ordinary skill can easily determine optimum dosages, dosing methodologies and repetition rates. Optimum dosages can vary depending on the relative potency of individual pharmaceutical compositions and can generally be estimated based on EC₅₀s found to be effective in in vitro and in vivo animal models.

D. Combination Therapies and Procedures

In some embodiments, compositions of dendrimers conjugated or complexed with one or more small molecule inhibitors of neutral sphingomyelinase 2 and/or additional therapeutic or diagnostic agents are administered in combination with one or more conventional therapies, for example, a conventional cancer, anti-infectious agent or antiinflammatory therapy. In some embodiments, the conventional therapy includes administration of one or more of the compositions in combination with one or more additional active agents. The combination therapies can include administration of the active agents together in the same admixture, or in separate admixtures. Therefore, in some embodiments, the pharmaceutical composition includes two, three, or more active agents. Such formulations typically include an effective amount of an immunomodulatory agent targeting tumor microenvironment. The additional active agent(s) can have the same, or different mechanisms of action. In some embodiments, the combination results in an additive effect on the treatment of the cancer. In some embodiments, the combinations result in a more than additive effect on the treatment of the disease or disorder.

In some embodiments, the formulation is formulated for intravenous, subcutaneous, or intramuscular administration to the subject, or for enteral administration. In some embodiments, the formulation is administered prior to, in conjunction with, subsequent to, or in alternation with treatment with one or more additional therapies or procedures. In some embodiments the additional therapy is performed between drug cycles or during a drug holiday that is part of the dosage regime. For example, in some embodiments, the additional therapy or procedure is surgery, a radiation therapy, or chemotherapy. Examples of preferred additional therapeutic agents include other conventional therapies known in the art for treating the desired disease, disorder or condition.

In the context of Alzheimer's disease, the other therapeutic agents can include one or more of acetylcholinesterase inhibitors (such as tacrine, rivastigmine, galantamine or donepezil), beta-secretase inhibitors such as JNJ-54861911, antibodies such as aducanumab, agonists for the 5-HT2A receptor such as pimavanserin, sargramostim, AADvac1, CAD106, CNP520, gantenerumab, solanezumab, and memantine.

In the context of Dementia with Lewy Bodies, the other therapeutic agents can include one or more of acetylcholinesterase inhibitors such as tacrine, rivastigmine, galantamine or donepezil; the N-methyl d-aspartate receptor antagonist memantine; dopaminergic therapy, for example, levodopa or selegiline; antipsychotics such as olanzapine or clozapine; REM disorder therapies such as clonazepam, melatonin, or quetiapine; anti-depression and antianxiety therapies such as selective serotonin reuptake inhibitors (citalopram, escitalopram, sertraline, paroxetine, etc.) or serotonin and noradrenaline reuptake inhibitors (venlafaxine, mirtazapine, and bupropion) (see, e.g., Macijauskiene, et al., Medicina (Kaunas), 48(1):1-8 (2012)).

Exemplary neuroprotective agents are also known in the art in include, for example, glutamate antagonists, antioxidants, and NMDA receptor stimulants. Other neuroprotective agents and treatments include caspase inhibitors, trophic factors, anti-protein aggregation agents, therapeutic hypothermia, and erythropoietin.

Other common active agents for treating neurological dysfunction include amantadine and anticholinergics for treating motor symptoms, clozapine for treating psychosis, cholinesterase inhibitors for treating dementia, and modafinil for treating daytime sleepiness.

In the context of cancer treatment, the other therapies include one or more of conventional chemotherapy, inhibition of checkpoint proteins, adoptive T cell therapy, radiation therapy, and surgical removal of tumors.

In some embodiments, the compositions and methods are used prior to, in conjunction with, subsequent to, or in alternation with treatment with an immunotherapy such inhibition of checkpoint proteins such as components of the PD-1/PD-L1 axis or CD28-CTLA-4 axis using one or more immune checkpoint modulators (e.g., PD-1 antagonists, PD-1 ligand antagonists, and CTLA4 antagonists), adoptive T cell therapy, and/or a cancer vaccine. Exemplary immune checkpoint modulators used in immunotherapy include Pembrolizumab (anti-PD1 mAb), Durvalumab (anti-PDL1 mAb), PDR001 (anti-PD1 mAb), Atezolizumab (anti-PDL1 mAb), Nivolumab (anti-PD1 mAb), Tremelimumab (anti-CTLA4 mAb), Avelumab (anti-PDL1 mAb), and RG7876 (CD40 agonist mAb). In particular embodiments, the compositions and methods are used in alternation with treatment with an immunotherapy using PD-L1 antagonists.

In some embodiments, the compositions and methods are used prior to, in conjunction with, subsequent to, or in alternation with treatment with adoptive T cell therapy. In some embodiments, the T cells express a chimeric antigen receptor (CARs, CAR T cells, or CARTs). Artificial T cell receptors are engineered receptors, which graft a particular specificity onto an immune effector cell. Typically, these receptors are used to graft the specificity of a monoclonal antibody onto a T cell and can be engineered to target virtually any tumor associated antigen. First generation CARs typically had the intracellular domain from the CD3 ζ-chain, which is the primary transmitter of signals from endogenous TCRs. Second generation CARs add intracellular signaling domains from various costimulatory protein receptors (e.g., CD28, 41BB, ICOS) to the cytoplasmic tail of the CAR to provide additional signals to the T cell, and third generation CARs combine multiple signaling domains, such as CD3z-CD28-41BB or CD3z-CD28-OX40, to further enhance effectiveness.

In some embodiments, the compositions and methods are used prior to, in conjunction with, subsequent to, or in alternation with treatment with a cancer vaccine, for example, a dendritic cell cancer vaccine. Vaccination typically includes administering a subject an antigen (e.g., a cancer antigen) together with an adjuvant to elicit therapeutic T cells in vivo. In some embodiments, the cancer vaccine is a dendritic cell cancer vaccine in which the antigen delivered by dendritic cells primed ex vivo to present the cancer antigen. Examples include PROVENGE® (sipuleucel-T), which is a dendritic cell-based vaccine for the treatment of prostate cancer (Ledford, et al., Nature, 519, 17-18 (5 Mar. 2015). Such vaccines and other compositions and methods for immunotherapy are reviewed in Palucka, et al., Nature Reviews Cancer, 12, 265-277 (April 2012).

In some embodiments, the compositions and methods are used prior to or in conjunction with, or subsequent to surgical removal of tumors, for example, in preventing primary tumor metastasis. In some embodiments, the compositions and methods are used to enhance body's own anti-tumor immune functions.

E. Controls

The therapeutic result of the dendrimer complex compositions including one or more agents can be compared to a control. Suitable controls are known in the art and include, for example, an untreated subject or untreated cells or the same individual prior to treatment.

### VI. Kits

The compositions can be packaged in kit. The kit can include a single dose or a plurality of doses of a composition including one or more inhibitors of neutral sphingomyelinase 2 encapsulated in, associated with, or conjugated to a dendrimer, and instructions for administering the compositions. Specifically, the instructions direct that an effective amount of the composition be administered to an individual with a particular neurological disease, defect or impairment as indicated. The composition can be formulated as described above with reference to a particular treatment method and can be packaged in any convenient manner

The present invention will be further understood by reference to the following non-limiting examples.

### EXAMPLES

### Example 1

### Treated of AD with Neutral Sphingomyelinase2 (nSMase2) Inhibitors

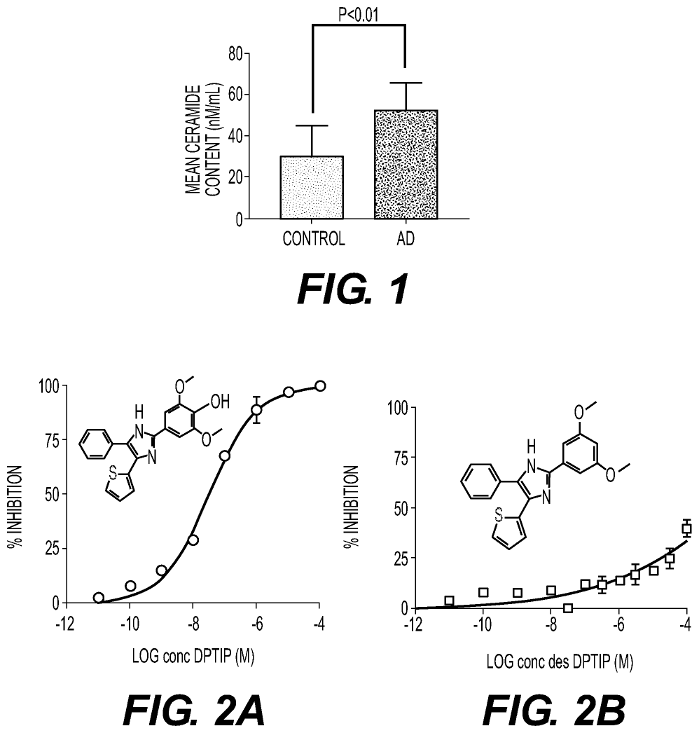

Ceramide levels in the cerebrospinal fluid (CSF) of AD patients have been shown to be significantly higher than those of patients with other neurological diseases, represented as control (FIG. 2). Moreover, immunohistochemistry studies showed that ceramide is aberrantly expressed in glia from postmortem AD brains, but not control brains. Double-labeling immunohistochemistry shows a regional coexistence of ceramide and Aβ-plaques. More recent studies indicate that the very long-chain plasma ceramides (C22:0 and C24:0) are altered in mild cognitive impairment (MCI) subjects along with predicted memory loss and decreased right hippocampal volume. A separate longitudinal study, where 99 women aged 70-79 without dementia monitored over a period of 9 years, revealed that higher baseline serum ceramides were associated with an increased risk of progression to AD. These findings show that ceramide quantification in plasma and activated glia could serve as biomarkers of AD progression.

Neutral Sphingomyelinase2 (nSMase2) is an important player in AD etiology. However, the currently available nSMase2 inhibitors are inadequate to develop potential treatments, and new nSMase2 inhibitors are being developed and tested. The nSMase2 inhibitor GW4869 has been employed as a test compound to conduct proof of concept studies. It has been used in chronic studies with no overt behavioral or physiological toxicities or body mass changes, supporting the potential of nSMase2 inhibition as a tolerable therapeutic approach. However, GW4869 is not potent at μM concentrations, exhibits very poor physiochemical properties including poor solubility (even in DMSO at 0.2 mg/mL) due to its highly lipophilic nature. Although identified over a decade ago, no analogs with improved potency or solubility have been described.

Methods

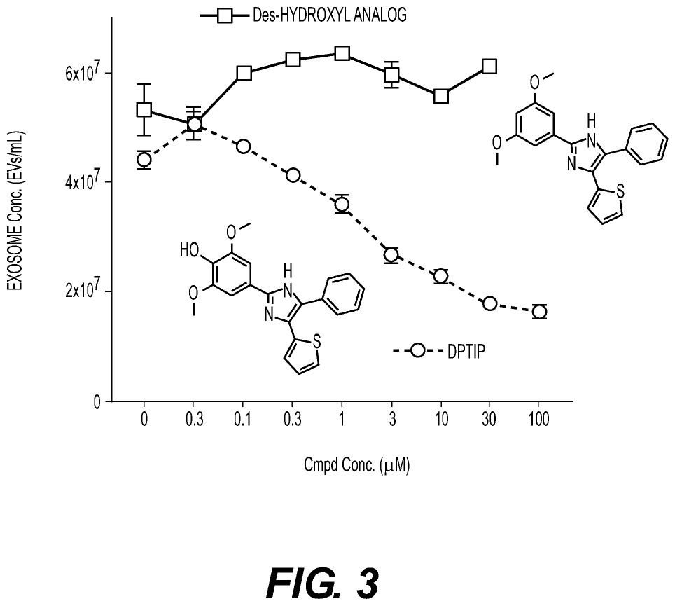

Following initial pilot screens that identified cambinol as nSMase2 inhibitor, a human nSMase2 high throughput screen (HTS) of >350,000 compounds was carried out using an enzyme coupled fluorescence-based human nSMase2 assay. Filtration of hit compounds using counter assay and drug likeness parameters lead to the discovery of 2,6-dimethoxy-4-(5-phenyl-4-(thiophen-2-yl)-1H-imidazol -2-yl) phenol (DPTIP) as the most promising compound, based on potency and chemical optimization feasibility. The IC50 for DPTIP was 30 nM (FIG. 3A). This IC50 is 30-fold and 160-fold more potent than the prototype inhibitors GW4869 (1 μM) and cambinol (5 μM), respectively. This is the first nSMase2 inhibitor described with nanomolar potency.

A des-hydroxyl analog of DPTIP was also synthesized to establish the significance of the hydroxyl group for inhibitory activity and it was shown to be inactive against human nSMase2 (IC50>100 μM) (FIG. 3B). This compound was then used as a structurally similar inactive DPTIP analog for comparison in subsequent pharmacological assays.

DPTIP is selective and does not inhibit members of two related enzyme families including alkaline phosphatase (IC50>100 μM), a phosphomonoesterase, or acid sphingomyelinase (IC50>100 μM), a phosphodiesterase closely related to nSMase2. Also, DPTIP has been screened in 759 bioassays at the National Center for Advancing Translational Sciences (NCATS) and only weak activity (μM) was observed in <2.5% of these assays (https://pubchem.ncbi.nlm.nih.gov/compound/5446044). DPTIP kinase profiling against the p38 kinases was conducted due to structural similarity of DPTIP to other p38 inhibitors.

Results

As shown in Table 1, below, DPTIP did not show inhibition of any of the four p38 kinases at concentrations of 0.001-100 μM (IC50 not quantifiable). Positive controls SB202190 and staurosporine showed potent inhibition of the respective p38 Map kinases.

TABLE 1
Profiling of DPTIP against p38 MAP Kinases

| | Compound | IC50 (M) | |
|---|---|---|---|
| | IC50 (M) | Control | Controll |
| Kinase | DPTIP | Cmpd | Cmpd ID |
| P38a/MAPK14 | | 2.63E−08 | SB202190 |
| P38b/MAPK11 | | 2.26E−08 | SB202190 |
| P38d/MAPK13 | | 1.87E−07 | Staurosporine |
| P38g | | 1.19E−07 | Staurosporine |

### Example 2

### In Vitro Inhibition of Exosome Release from Glial Cells

Methods

The ability of DPTIP to inhibit the release of exosomes from glial cells was evaluated in vitro. Mouse primary glia were activated by FBS and treated with DPTIP or its closely related inactive des-hydroxyl analog, at a concentration range of 0.03-100 μM using DMSO (0.02%) as vehicle control. Two hours after treatment, exosomes were isolated from the media and quantified by nanoparticle tracking analysis.

Results

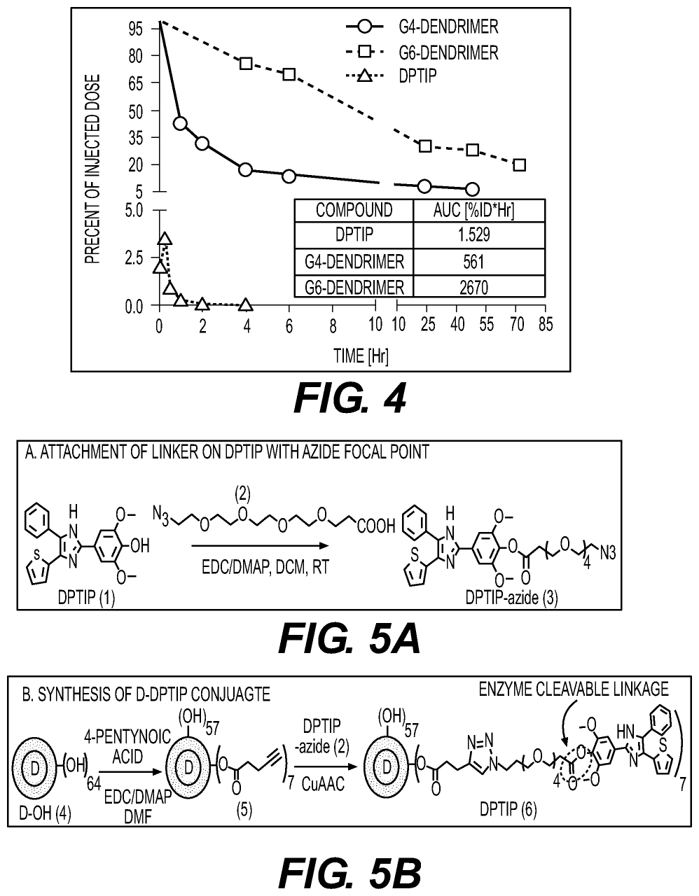

DPTIP inhibited exosome release in a dose-dependent manner (FIG. 4). In contrast, a closely related inactive analog had no effect, supporting the mechanism of DPTIP exosome inhibition happens via nSMase2.

GW4869 (a known nSMase2 inhibitor) showed significant changes in synaptic proteins, including increased post-synaptic protein PSD-95, increased NMDA receptor subunit NR2A, as well as the AMPA receptor subunit GluR1. Similar pilot studies with DPTIP (10 mg/kg daily; intraperitoneal) showed no significant effect on PSD95 or NR2A. Voltage traces measuring neuronal function were recorded from 300 μm horizontal brain slices on multielectrode array (MEA) plates continuously perfused with oxygenated artificial cerebrospinal fluid (ACSF) pre and post 10 μM DPTIP treatment. No significant differences in traces were observed, suggesting no effect on neuronal function following nSMase2 inhibition with DPTIP.

### Example 3

### Pharmacokinetics and Bioavailability of DPTIP and its Analogues

Methods

The in vitro and in vivo pharmacokinetic properties of DPTIP were evaluated.

Results

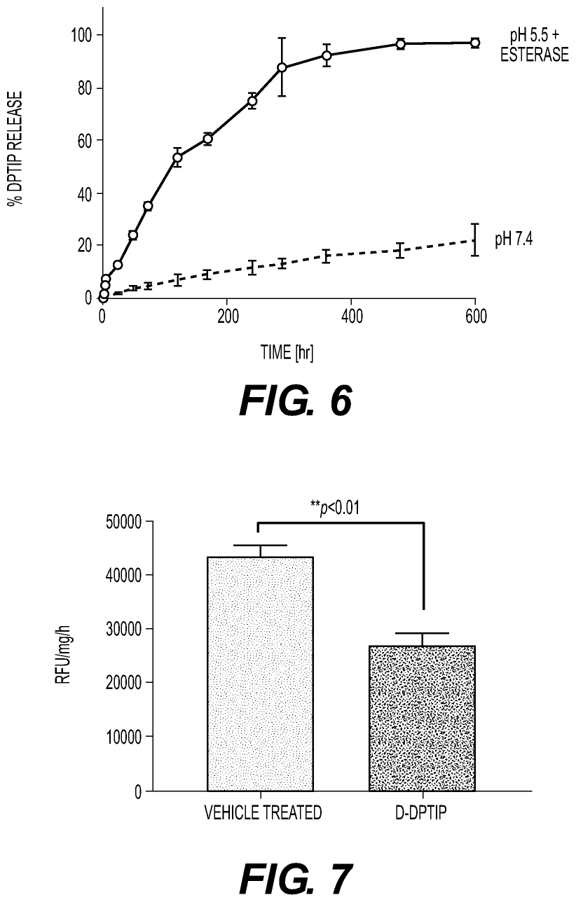

Phase I metabolic stability studies in mouse and human liver microsomes showed that DPTIP was completely stable (100% remaining at 1 hr) to CYP-dependent oxidation. This was encouraging as DPTIP contains the “thiophene” ring that can form reactive metabolites (e.g. thiophene-S oxides, thiophene epoxides) via Phase I oxidation reactions. In addition, DPTIP was also modestly stable to phase II glucuronidation (>50% remaining at 1 hr). However, the oral bioavailability and brain penetration following peroral (10 mg/kg PO; FIG. 6C) administration in mice were not optimal (F<5%, AUCbrain/plasma ratio <0.2). Further, the levels of DPTIP were undetectable 2-he post-administration due to rapid clearance (Clapp=92 mL/min/kg) and a short plasma half-life (t½=˜0.5 h). These results were confirmed in AD mice (3×Tg) following DPTIP (10 mg/kg i.p.) administration. AD mice exhibited poor brain DPTIP penetration (<0.2) comparable to wild-type (WT) mice.

Given DPTIP's poor oral bioavailability (F<5%) and limited brain penetration (B/P ratio <0.2) and fast clearance (plasma half-life t½=˜0.5 h), an extensive SAR effort (>200 analogs synthesized) was carried out to improve its pharmacokinetic properties. Even though it was possible to identify the parts of the pharmacophore that were important for inhibitory activity and to synthesize analogs with similar potency, it was not possible to identify analogs with improved oral bioavailability or clearance.

### Example 4

### Use of Hydroxyl PAMAM Dendrimers Improves Delivery and Retention of Small Molecules

Hydroxyl PAMAM dendrimers are nontoxic, even at multiple doses of >500 mg/kg in preclinical models and are cleared intact (unmetabolized) through the kidney, including humans. These dendrimers, without any targeting ligand, selectively localize in activated glia in the brain and can deliver drugs to the site of injury producing positive therapeutic outcomes. Importantly, no such cellular uptake is observed in the healthy control animals. The mechanism for this selective uptake has not been seen with other types of nanoparticles. The dendrimer is able to cross the impaired BBB and diffuse rapidly in the brain tissue for eventual uptake by increasingly endocytic activated glia. Dendrimer-N-acetyl cysteine at 10 mg/kg drug (oral or IV) showed significant therapeutic benefit in motor function, reduction in neuroinflammation, oxidative stress, and neurologic injury. This compound is undergoing clinical trials after successful GMP production and toxicity studies and has completed healthy adult volunteer studies. Multiple studies using small and large animal models of brain injury have demonstrated that hydroxyl-terminated dendrimers cross impaired BBB in multiple species targeting injured.

Methods

The effect of dendrimer size on brain uptake was examined and the pharmacokinetics in both canine and rodent models of brain injury using generation 6 (G6, ˜6.7 nm, ˜56 kDa) and generation 4 (G4, ˜4.3 nm, ˜14 kDa) PAMAM dendrimers with Cy5 labeling and fluorescence quantification were investigated.

G6 dendrimer has extended plasma circulation times (plasma half-life, t½˜24 h, ˜30% of the injected dose at 72 h; FIG. 4) compared to G4 dendrimers (t½˜6 h, ˜5% of the injected dose at 72 h). This is accompanied by a ˜10-fold increase in the brain AUC for the G6 dendrimer (Mishra M K, et al., ACS nano. 2014; Zhang F, et al., Journal of Controlled Release. 249:173-82 (2017)). In rats, the AUC of G6 dendrimer was also ˜10 fold more than the G4 dendrimer. Although not a direct comparison, plasma AUCs of either of the dendrimers are significantly higher than that of DPTIP following systemic administration due to enhanced circulation time afforded by the dendrimers. In addition, DPTIP has a short plasma half-life (t½=˜0.5 h) versus G4/G6 dendrimers (t½=6-24 h).

A pilot scale synthesis was conducted to confirm if DPTIP can be conjugated with dendrimers. The synthesis of D-DPTIP was achieved using highly efficient copper (I) catalyzed alkyne-azide click (CuAAC) chemistry (Franc G and Kakkar A. Chemical Communications. 2008 (42):5267-76) The synthesis began with the modification of DPTIP to attach an orthogonal linker with azide terminal through cleavable ester bond (FIG. 5A). The purpose of the azide group is to participate in CuAAC reaction with the alkyne functions on the surface of the dendrimer. The hydroxyl group in DPTIP (1) was reacted with azido-PEG4-acid (2) in the presence of N-(3-dimethylaminopropyl)-N′-ethylcarbodiimide hydrochloride (EDC) and 4-(dimethylamino)pyridine (DMAP), as coupling agents. The crude product was purified using column chromatography to obtain DPTIP-azide (3). The product (3) was characterized via NMR, mass and HPLC techniques. On the other hand, dendrimer surface was modified to attach a linker bearing complimentary alkyne groups (FIG. 5B). The as-received generation 4 hydroxyl PAMAM dendrimer (D-OH; 4) was purified by dialysis, centrifugal filtration, and semi-preparative HPLC fractionation techniques previously established to remove dimers/trailing generations. The purified D-OH was reacted with pentynoic acid in the presence of coupling agents to obtain partially alkyne-terminated dendrimer (5) with 7 linkers attached. Finally, the CuAAC click reaction was performed between dendrimer (5) and DPTIP-azide (3) to obtain D-DPTIP conjugate with 7 drug molecules attached. The final conjugate was purified by dialysis. All the intermediates and the final conjugate were characterized using 1H-NMR, 13C-NMR, HPLC and MALDI-TOF MS analyses. The release of free drug through cleavable ester linkage was analyzed by HPLC.

### Results

On an average, seven drug molecules were conjugated to the dendrimer (13% weight), as calculated using 1H NMR comparing the integration of dendrimer amidic protons to ester methylene protons and triazole ring protons. In vitro drug release was analyzed in the presence of esterase (pH 5.5) at physiological temperature. D-DPTIP showed >80% drug release over a period of approximately ten days (FIG. 6). Conjugation of a variety of therapeutic molecules on the periphery of dendrimers can be carried out using methods known to those skilled in the art.

### Example 5

### Orally Administered Cy5-Dendrimer-DPTIP to AD Mice is Delivered to Brain Glial Cells and Shows Target Engagement by Significant Inhibition of nSMase2 Activity

Methods

The in vivo uptake and retention of orally administered Cy5-D-DPTIP conjugate in activated microglia in AD mice was assessed using fluorescence spectroscopy. In tandem, target engagement employing functional nSMase2 inhibition in isolated CD11b+ cells were performed. In brief, 9-month-old P301S AD mice were dosed with Cy5-D-DPTIP and sacrificed 24 h (for imaging) and 96 h (for target engagement) later via a transcardial perfusion of ice-cold PBS. For imaging, brains were post-fixed in 10% formalin for 48 h at 4° C., flash-frozen, stored at −80° C., sectioned at a thickness of 30 μm, and stained for CD11b+ cells (Iba1) and DAPI. For target engagement, glial cells were isolated from fresh brains.

Results

Activated microglia in AD mouse brains selectively engulfed Cy5-D-DPTIP. Positive Cy5 signal was observed near the dentate gyrus region of the hippocampal formation in brains of AD mice. The positive Cy5 signal overlapped with Iba-1 staining, indicating uptake in activated microglia cells; moreover, significant inhibition of nSMase2 activity was observed in glial cells treated with D-DPTIP signal (FIG. 7).

### Example 6

### Orally Administered Cy5-Dendrimer-DPTIP to PS19 Mice Selectively Accumulates in Brain Tissue

Methods

The dose-dependent pharmacokinetics of D-DPTIP was evaluated in 3- to 4-month-old PS19 mice (The Jackson Laboratory, Stock No. 008169). D-DPTIP was dosed (10, 30 and 100 mg/kg free drug equivalent; 10 mL/kg) via oral gavage to the PS19 mice. At predetermined time points (24, 72, 120 hours post-administration) animals were euthanized, and brain tissues were harvested following blood collection. Plasma was generated from blood by low-speed centrifugation (3000 g). Plasma and brain tissue were immediately snap frozen in liquid nitrogen and stored at ˜80° C. for DPTIP quantification by LC-MS/MS Bioanalysis: Brain samples were homogenized in PBS and incubated with 2 mg/mL liver CES enzyme for 60 min to ensure the release of DPTIP from the D-DPTIP present in the brain. Calibration standards (1 nM-10,000 nM) for brain were prepared by spiking DPTIP in brain homogenates (in PBS). For plasma quantification DPTIP calibration standards (1 nM-10,000 nM) were prepared using naïve mouse plasma spiked with DPTIP. DPTIP standards and samples were extracted from plasma and brain by one-step protein precipitation using acetonitrile (100% v/v) containing internal standard (losartan—0.5 μM). The samples were vortex-mixed and centrifuged (14,000 rpm for 10 min at 4° C.) and the supernatant was analyzed for DPTIP using LC-MS/MS as described previously (Rojas C, et al., Sci Rep. 2018 Dec. 7; 8(1):17715).

Results

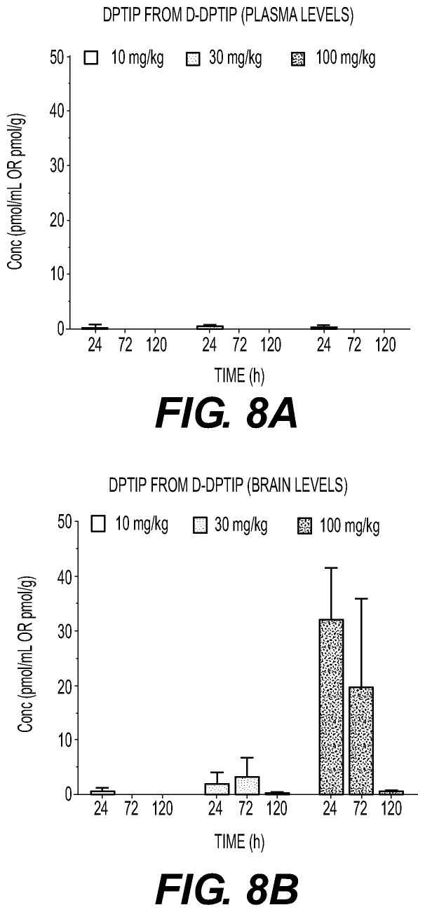

D-DPTIP conjugate (10, 30, and 100 mg/kg DPTIP equivalent) was dosed via oral gavage and DPTIP release was measured at 24, 72 and 120 hours post dose. In plasma, D-DPTIP showed no measurable levels of DPTIP at any timepoint or dose level (FIG. 8A). In contrast, 100 mg/kg dose provided brain concentrations of DPTIP at its nSMase2 IC50 (20-35 nM) up to 72 hours (FIG. 8B). However, D-DPTIP showed no measurable brain concentrations of DPTIP at 120 hours for any dose level.

### Example 7

### Orally Administered Cy5-Dendrimer-DPTIP to PS19 Mice Selectively Inhibits nSMase2 Activity in Activated Microglia

Methods

For target engagement evaluation 3- to 4-month-old PS19 mice were dosed with D-DPTIP orally (10 and 100 mg/kg) and sacrificed at 72 hours post administration. Microglial (CD11b+) cells were isolated from whole brains according to a previously described method with minor modification (Zhu X, et al., Neuropsychopharmacology. 2019 March; 44(4):683-694), and nSmase2 activity was measured using a fluorescent assay (Figuera-Losada M, et al., PLoS One. 2015 May 26; 10(5):e0124481). Imaging studies were also performed in isolated glial cells using fluorescently tagged Dendrimer-CY5-DPTIP to confirm microglia accumulation of D-DPTIP.

Results

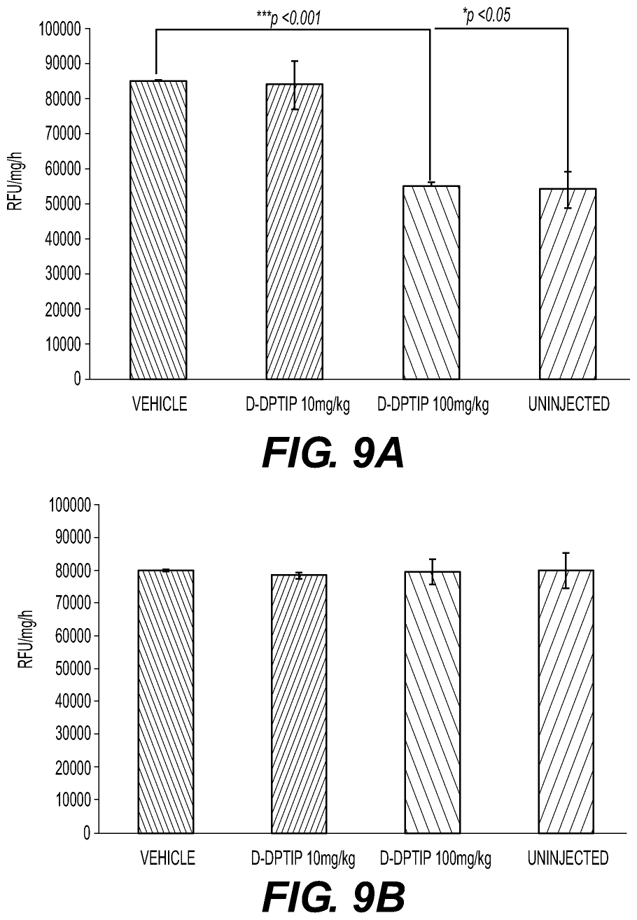

Oral D-DPTIP significantly inhibited microglial nSMase2 activity at 72 hours post administration with 100 mg/kg dose but no inhibition was observed at 10 mg/kg (FIG. 9A). No inhibition was observed in non-microglial cells (FIG. 9B), suggesting specific targeting to microglia from D-DPTIP. In addition, it was observed that activated microglia in AD mouse brains selectively engulfed Cy5-D-DPTIP. Positive Cy5 signal was observed near the dentate gyms region of the hippocampal formation in brains of AD mice. The positive Cy5 signal overlapped with Iba-1 staining, indicating microglia uptake.

### Example 8

### Tumor Growth and Survival Experiments

Methods

Six- to eight-week-old male C57BL/6mice were used for this study. All mouse procedures were approved by the Johns Hopkins University Institutional Animal Care and Use Committee. MC38 cells were cultured in DMEM supplemented with 10% FBS, 2 mM glutamine, 1% penicillin/streptomycin, and 10 mM HEPES. Cell line were regularly tested to confirm mycoplasma free using a Myco Alert mycoplasma detection kit (Lonza). Cells were kept in culture no longer than 3 weeks. MC38 (5×10⁵ cells in 200 μl per mouse) cells were subcutaneously (s.c.) inoculated into right flank of C57BL/6J mice. Groups were randomized based on tumor size on the day of beginning treatment. Mice was administered (treated) by i.p. injection with Isotype Control (200 μg/mice) or Anti-PDL1 (200 μg/mice) on day 12, 15 and 18 respectively or D-DPTIP Control (300 μl/mice) or in combination with Anti-PDL1 (200 μg/mice) and D-DPTIP (2.3 mg/mouse) on every alternative day. Tumor burdens were monitored every 2-4 days by measuring length and width of tumor. Tumor volume was calculated using the formula for caliper measurements: tumor volume=(L×W2)/2, where L is tumor length and is the longer of the 2 measurements and W is tumor width, tumor area=L×W. Mice were euthanized when tumor size exceeded 2 cm in any dimension or when the mice displayed hunched posture, ruffled coat, neurological symptoms, severe weight loss, labored breathing, weakness or pain.

For G6 D-DPTIP pharmacokinetics in mice inoculated with EL4 lymphoma, naïve male and female C57BL/6 mice (weighing between 25-30 g) at 6-8 weeks of age, were used. The animals were maintained on a 12 h light-dark cycle with ad libitum access to food and water. EL4 mouse lymphoma cells upon confluence were injected s.c. (0.3×10⁶ cells) and tumor growth was monitored. Tumor volume was calculated using the formula V=(L×W)/2, where V is tumor volume, W is tumor width, and L is tumor length and mice with a mean tumor volume around 400 mm3 were considered for the pharmacokinetic study (n=3 mice per time-point, 2 males and 1 female). Animals were dosed with 10 mg/kg DPTIP equivalent of G6-DPTIP and plasma and tumors were collected at various time point. Plasma and tumor samples were analysed for DPTIP using LC/MS-MS.

Results

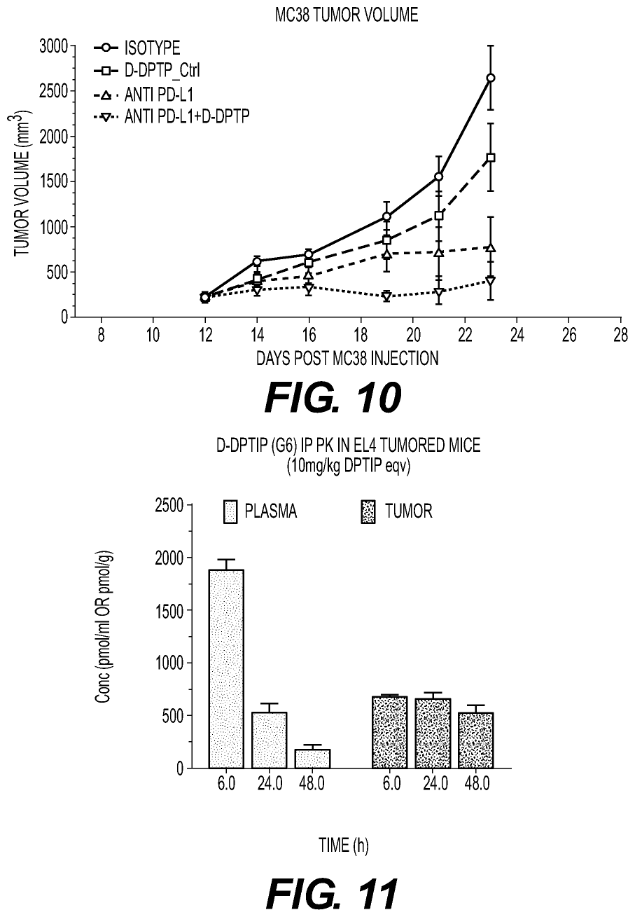

Monotherapies with -DPTIP or Anti-PDL1 alone in MC38 tumor model showed significant inhibitory effect on tumor growth when compared to isotype control. Combination therapy of D-DPTIP with Anti-PDL1 showed the greatest inhibitory effect on tumor growth when compared to respective monotherapies with -DPTIP or Anti-PDL1 alone in MC38 tumor model (FIG. 10).

For G6 D-DPTIP pharmacokinetics in mice inoculated with EL4 lymphoma, G6-DPTIP showed excellent pharmacokinetics with detectable levels in plasma and tumors up to 48 hr post administration. Notably, G6-D-DPTIP afforded sustained tumor levels at >400 nM (˜13 fold IC50) up to 48 hr post administration (FIG. 11).

### Example 9

### D-DPTIP Treatment Reduced Tau Propagation to Neurons of the Contralateral Dentate Gyrus

Methods

All mouse procedures were approved by the Johns Hopkins University Institutional Animal Care and Use Committee. 10-week old male C57BL6/J wild type mice were stereotaxically injected with 6×10¹² viral particles of AAV1-CBA-P301L/S320F hTau-WPRE vector into the left hippocampus (coordinates, from Bregma: AP: −2.35; ML: −2.10; DV: −1.85). Mice were given two days to rest following the surgery before treatment began with either empty dendrimer vehicle (n=9) or 769 mg/kg D-DPTIP (100 mg/kg DPTIP eq dose) (n=8) PO twice weekly for 6 weeks. After 6 weeks, mice were deeply anesthetized with isoflurane before being transcardially perfused with 1× PBS followed by 2% paraformaldehyde to fix the tissue for imaging studies. The brains were then cryoprotected in 30% sucrose before being cryosectioned at 30 μm. The sections were blocked and permeabilized for 1 h at room temperature with 5% normal goat serum in 1× PBS+0.1% Triton X-100. The sections were incubated overnight at 4° C. with primary antibodies against neurons (NeuN) to confirm tau positivity is in the neurons and phosphorylated tau (pThr181) before being incubated with appropriate fluorophore conjugated secondary antibodies. Images were taken using a Zeiss LSM800 confocal microscope with identical settings used for all image acquisition ensuring that no pixels were saturated. A single focal plane was imaged where the pThr181 hTau fluorescence signal was at its maximum and 8-10 images were taken from each mouse at the same hippocampal locations on both the injection and contralateral sides. Image acquisition and analysis was done blinded to treatment status. Raw TIFF images were used for mean fluorescence intensity (MFI) quantification comparing vehicle (n=6) and D-DPTIP (n=4) treated mice. Using ImageJ software, the ipsilateral pyramidal layer of the dentate gyrus was traced in each image and the MFI of the pThr181 hTau signal was determined. The MFI of the hTau signal on the contralateral side was determined over the entire image to account for axonal and cell body tau signal. To account for variability in AAV injection volume, uptake, and expression levels, we took the ratio between the contralateral and ipsilateral MFI. A mixed effects two-way ANOVA was used to determine statistical significance using Prism statistical software. Three vehicle treated and four D-DPTIP treated animals were removed from the study due to improper injection location.

Results

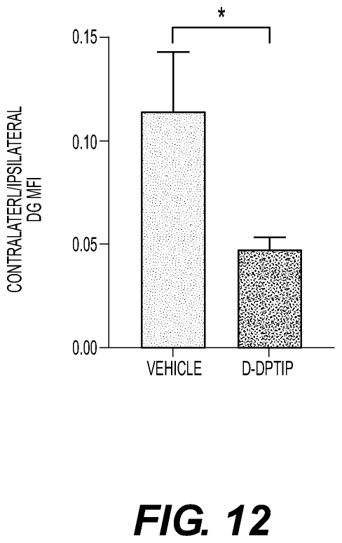

Six weeks following treatment initiation, empty dendrimer vehicle treated mice had neuronal Thr181 phosphorylated tau signal in the hilus region of the contralateral DG while the D-DPTIP treated animals had lower tau signal in the same region. Quantification of the contralateral/ipsilateral MFI in the hilus region of the DG showed a 2.4-fold reduction in the D-DPTIP treated animals (FIG. 12; vehicle=0.1144, n=57 images/6 mice; D-DPTIP=0.0475, n=40 images/4 mice; p=0.0344).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A composition comprising dendrimers complexed, covalently conjugated, or intra-molecularly dispersed or encapsulated with one or more therapeutic or prophylactic agents that decrease exosome secretion, reduce Aβ plaque formation, reduce tau propagation, improve cognition, or combinations thereof. for the treatment of neurological disease, cancer, infectious disease, or inflammation associated therewith

2. The composition of claim 1, wherein the agents inhibit or reduce activity and/or quantity of neutral sphingomyelinase 2.

3. The composition of claim 1, wherein the agents are small molecule inhibitors of neutral sphingomyelinase 2.

4. The composition of claim 3, wherein the one or more small molecule inhibitors of neutral sphingomyelinase 2 are selected from the group consisting of 2,6-dimethoxy-4-(5-phenyl-4-(thiophen-2-yl)-1H-imidazol-2-yl) phenol (DPTIP), phenyl(R)-(1-(3-(3,4-dimethoxyphenyl)-2,6-dimethylimidazo[1,2-b]pyridazin-8-yl)pyrrolidin-3-yl)-carbamate (PDDC), N,N′-Bis[4-(4,5-dihydro-1H-imidazol-2-yl)phenyl]-3,3′-p-phenylene-bis-acrylamide dihydrochloride (GW4869), cambinol, 4-(4,5-diisopropyl-1H-imidazol-2-yl)-2,6-dimethoxyphenol, and derivatives and analogs thereof.

5. The composition of claim 3, wherein the inhibitor of neutral sphingomyelinase 2 is DPTIP, or a derivative or analog thereof.

6. The composition of claim 1, wherein the dendrimers are generation 4, generation 5, generation 6, generation 7, or generation 8 dendrimers.

7. The composition of claim 1, wherein the dendrimers are poly(amidoamine) (PAMAM) dendrimers.

8. The composition of claim 1, wherein the dendrimers are hydroxyl-terminated PAMAM dendrimers.

9. The composition of claim 1, wherein the dendrimers are covalently conjugated to the one or more therapeutic or prophylactic agents.

10. A pharmaceutical composition comprising the composition of claim 1 and one or more pharmaceutically acceptable excipients.

11. The pharmaceutical composition of claim 10 formulated for parenteral or oral administration.

12. The pharmaceutical composition of claim 10 in a form selected from the group consisting of hydrogels, nanoparticle or microparticles, suspensions, powders, tablets, capsules, and solutions.

13. A method for reducing the quantity of brain and/or serum exosomes, brain and/or serum ceramide levels, serum anti-ceramide IgG, glial activation, total Aβ42 and plaque burden, tau phosphorylation, improving cognition, or combinations thereof, in a subject comprising systemically administering to the subject an effective amount of the composition of claim 1.

14. The method of claim 13 for treating Alzheimer's disease or dementia in a subject comprising systemically administering to the subject an effective amount of the composition of claim 1 to treat, alleviate, and/or prevent one or more symptoms associated with Alzheimer's disease or dementia.

15. The method of claim 13, wherein the composition is administered in an effective amount to decrease exosome secretion in the brain, reduce Aβ plaque formation and/or tau propagation in the brain, improve cognition, or combinations thereof.

16. The method of claim 13, wherein the composition is administered in an effective amount to inhibit or reduce activity and/or quantity of neutral sphingomyelinase 2 in activated microglia.

17. The method of claim 13, wherein the composition is administered in an effective amount to reduce the concentration of ceramide in the cerebrospinal fluid and/or serum of the subject.

18. The method of claim 13, wherein the composition is in an effective amount to reduce the quantity of exosomes in the brain of the subject.

19. The method of claim 13, wherein the subject has an increased level of ceramide in the cerebrospinal fluid and/or serum, compared to a healthy control subject.

20. The method of claim 13 for inhibiting activities of neutral sphingomyelinase 2 in activated microglia in the brain of a subject comprising systemically administering to the subject an effective amount of the composition of claim 1.

21. The method of claim 13 for increasing generation of new neurons, or reducing or preventing the rate of neuron loss in a subject comprising systemically administering to the subject an effective amount of the composition of claim 1.

22. The method of claim 13 for increasing the hippocampal volume, or reducing or preventing the rate of decrease of hippocampal volume of a subject comprising systemically administering to the subject an effective amount of the composition of claim 1.

23. The method of claim 19, wherein the subject has an increased level of ceramide in the cerebrospinal fluid and/or serum, compared to a healthy control subject.

24. The method of claim 19, wherein the subject has Alzheimer's disease or dementia.

25. The method of claim 13, wherein the composition is administered orally or parenterally.

26. The method of claim 13, wherein the composition is administered intravenously.

27. A method of treating one or more symptoms of cancer, infectious disease or inflammation in a subject in need thereof comprising administering to the subject an effective amount of the composition of claim 1.

28. The method of claim 27, wherein the cancer is breast cancer, cervical cancer, ovarian cancer, uterine cancer, pancreatic cancer, skin cancer, multiple myeloma, prostate cancer, testicular germ cell tumor, brain cancer, oral cancer, esophagus cancer, lung cancer, liver cancer, renal cell cancer, colorectal cancer, duodenal cancer, gastric cancer, and colon cancer.

29. The method of claim 27, wherein the effective amount is effective to reduce tumor size or inhibit tumor growth.

30. The method of claim 27 further comprising administering to the subject one or more immune checkpoint modulators selected from the group consisting of PD-1 antagonists, PD-1 ligand antagonists, and CTLA4 antagonists.

31. The method of claim 27 further comprising administering to the subject adoptive T cell therapy, and/or a cancer vaccine.

32. The method of claim 27 further comprising performing surgery or radiation therapy to the subject.

33. The method of claim 27, wherein the composition is administered orally or parenterally.

34. The method of claim 27 for treating or alleviating one or more inflammatory diseases or disorders in a subject in need thereof comprising administering to the subject an effective amount of the composition of claim 1 to treat or alleviate one or more symptoms associated with the one or more inflammatory diseases or disorders.

35. The method of claim 34, wherein the one or more inflammatory diseases or disorders are selected from the group consisting of airway inflammation, allergic airway inflammation, atherosclerosis, cerebral ischemia, hepatic ischemia reperfusion injury, myocardial infarction, and sepsis.

36. The method of claim 34, wherein the composition is administered in an amount effective to suppress or inhibit one or more pro-inflammatory cells associated with the one or more inflammatory diseases or disorders.

37. The method of claim 34, wherein the pro-inflammatory cells are activated macrophages or microglia.

38. The method of claim 27 for treating or alleviating one or more bacterial, parasitic, fungal or viral infections in a subject in need thereof comprising administering to the subject an effective amount of the composition of claim 1 to treat or alleviate one or more symptoms associated with the one or more bacterial or viral infections.

39. The method of claim 38, wherein the one or more bacterial or viral infections are caused by one or more causative agents selected from the group consisting of human immunodeficiency virus (HIV), Zika virus, Hepatitis C, Hepatitis E, Rabies, Langat virus (LGTV), Dengue virus (DENV), cytomegalovirus (HCMV), and Newcastle disease virus (NDV), Epsilon-toxin from Clostridium perfringens, and shiga toxin from Escherichia coli.

40. The method of claim 39, wherein the one or more causative agents target or infect activated microglia and astrocytes.

41. The method of claim 38, wherein the composition is administered in an amount effective to reduce or inhibit viral replication, viral load, and/or viral release, or a combination thereof.

42. The method of claim 34, wherein the composition is administered orally or parenterally.
